(19) United States
(12) Patent Application Publication
(10) Pub. No.: US 2025/0388976 A1
(43) Pub. Date: Dec. 25, 2025

(54) A METHOD FOR DETECTING THE PRESENCE OF AT LEAST TWO PATHOGENS IN A SAMPLE

(71) Applicant: SEFUNDA AG, MUTTENZ (CH)

(72) Inventors: LAURIN DIENER, ZÜRICH (CH); ANNA KATHARINA ELMER, LÖRRACH (DE); MARION GILSDORF, NUSSHOF (CH); SEREJ D. LEY, BASEL (CH); MELANIE SCHÄPERS, LÖRRACH (DE); TOSONI DENIZ, SISSACH (CH)

(73) Assignee: SEFUNDA AG, MUTTENZ (CH)

(21) Appl. No.: 18/838,900

(22) PCT Filed: Feb. 17, 2023
(86) PCT No.: PCT/EP2023/025080

§ 371 Date: Aug. 15, 2024

(30) Foreign Application Priority Data
Feb. 17, 2022 (EP) 22157375.1

(51) Int. Cl. C12Q 1/689; B01L 3/00

(52) U.S. Cl. CPC C12Q 1/689; B01L 3/502753; B01L 2200/04; B01L 2200/16; B01L 2300/0681; B01L 2300/0816; C12Q 2600/106

(57) ABSTRACT

The invention relates to a method for detecting the presence of at least two pathogens in a sample, wherein additionally subtypes and/or variant-derived properties such as treatment susceptibility are determined. The method may be a polymerase chain reaction and may be facilitated by a cartridge and dried reagents. The pathogens may be bacterial pathogens such as Chlamydia trachomatis and Neisseria gonorrhoeae. The invention further relates to a cartridge and/or a cartridge reader for performing the method of the invention.

### CROSS-REFERENCE TO RELATED APPLICATIONS

This application is a U.S. National Phase Application under 35 U.S.C. § 371 of International Application No. PCT/EP2023/025080 filed Feb. 17, 2023, which claims the benefit of priority to European Patent Application No. 22157375.1 filed Feb. 17, 2022. The entire contents of the foregoing applications are hereby incorporated herein by reference.

### SEQUENCE LISTING

The instant application contains a Sequence Listing which has been submitted electronically in XML format and is hereby incorporated by reference in its entirety. Said XML copy, created on May 2, 2025, is named AD2327_US_BS_Replacement_Sequence_Listing.xml and is 57,344 bytes in size.

The invention relates to a method for detecting the presence of at least two pathogens in a sample, wherein additionally, subtypes and/or variant-derived properties such as treatment susceptibility are determined. The method may be a polymerase chain reaction and may be facilitated by a cartridge and dried reagents. The pathogens may be bacterial pathogens such as Chlamydia trachomatis and Neisseria gonorrhoeae. The invention further relates to a cartridge and/or a cartridge reader for performing the method of the invention.

The resurgence of infectious diseases will, along with climate change, be one of the major challenges for the global population in the coming years. The increase in bacterial infections that resist treatment with antibiotics will play just as important a role as new viral variants such as SARS-COV-2. Today empirical treatment according to a “best guess” principle continues to dominate. Specific, cost-effective and, above all, rapid on-site diagnostics have not prevailed in recent years, despite certain efforts by the industry. One reason for this may be the focus on only a certain aspect of the diagnostic test rather than integration and mass production of all components by many IVD startups.

Sexually transmitted infections (STI) are most common in vulnerable populations, who seek care in emergency rooms (ER) or urgent care clinics (UCC) or publicly funded clinics (U.S. Department of Health and Human Services, 2020, Sexually Transmitted Infections, National Strategic Plan for the United States 2021-2025). Chlamydia and gonorrhea (caused by the bacteria C. trachomatis and N. gonorrhoeae (CT/NG)) can spread easily and can initially be symptomless. If untreated these infections can cause long term effects including cervical cancer, preterm birth, ectopic pregnancy, neonatal encephalitis, and infertility (WHO, 2016, WHO guidelines for the treatment of Chlamydia trachomatis; WHO, 2016, WHO guidelines for the treatment of Neisseria gonorrhoeae; CDC, 2021, Treatment Guidelines Evidence N. gonorrhoeae.). Today most CT and NG infections can still be treated easily and effectively if diagnosed correctly and in time, but antimicrobial resistance (AMR) has become an increased threat especially for gonorrhea treatment. The first antimicrobial resistance in NG appeared in the mid-40 s of the last century. Since then, genetic resistance mechanisms to 6 antimicrobial families have emerged over the time in several NG variants (Unemo, Magnus and Lahra, Monica M. s. I . . . . The Lancet Microbe, 2021, Vol 2, Issue 11, pp. e627-e636; Unemo, Magnus, Del Rio, Carlos and Shafer, William, Microbiol Spectr. 2016 June; 4 (3)), limiting treatment options and forcing doctors to empirically prescribe the only effective drug as a last resort. Development of new antimicrobial agents is stagnant for several, mostly economic reasons, leaving no other choice than trying to keep the last available drug effective for as long as possible to avoid the spread of untreatable pan-resistant gonococci (Low, Nicola and Unemo, Magnus. s. l.: Ovid Technologies (Wolters Kluwer Health), 2016, Current Opinion in Infectious Diseases, Bd. 29, S. 45-51; CDC, 2021, Treatment Guidelines Evidence N. gonorrhoeae).

In most cases chlamydia infections are symptomless but certain strains (LGV 1-3) can cause Lymphogranuloma venereum an invasive infection of the lymph system and require a more rigorous antimicrobial treatment (WHO, 2016, WHO guidelines for the treatment of Chlamydia trachomatis).

Methods to detect CT and NG infections are known, but current methods do not offer extensive AMR detection and lack LGV differentiation. This can have a direct impact on treatment decision, which impacts the opportunity to catch the more dangerous variant of CT and resistant NG early on before they further spread, and to minimize unnecessary treatment of the less harmful CT variants.

To date there still exists a need in the art of combining pathogen identification with the most relevant variant-derived properties such as genetic antimicrobial resistance markers along with sub-species identification that allows clinicians to make informed treatment decisions leading to a safe and effective but also most considerate and preservative treatment for their patients at a low cost.

Thus, there is a need for improved tests providing faster and/or more accurate results.

The above technical problem is solved by the embodiments disclosed herein and as defined in the claims.

Accordingly, the invention relates to, inter alia, the following embodiments:

1. A method for determining whether a sample contains a pathogen, the method comprising the steps of:
a) separating the sample into at least two subsamples;
b) bringing different amplification reagents into contact with at least two of the subsamples for amplifying at least three different target nucleic acid sequences, wherein at least two of the target nucleic acid sequences are identification nucleic acid sequences, wherein each of the identification nucleic acid sequences is characteristic for a pathogen; and
   1.) at least one of the target nucleic acid sequences is a variant target sequence, wherein the variant target sequence is indicative of at least one mutation of at least one of the pathogens; and/or
   2.) at least one of the target nucleic acid sequences is an identity specifying target sequence, wherein at least one of the identification nucleic acid sequences is characteristic for a plurality of subgroups of at least one of the pathogens and the identity specifying target sequence is comprised in one of the subgroups of the pathogen;
c) determining the presence or absence of amplification products of the target nucleic acid sequences in the subsamples using at least one detection probe; and
d) determining whether the sample contains a pathogen, wherein presence of an amplification product is an indication of a presence, an absence and/or a quantity of one or more pathogens in the sample.

2. The method of embodiment 1, wherein separating the sample according to step
a) comprises the substeps of:
   1.) accepting a cartridge having a sample port assembly containing the sample;
   2.) enriching the nucleic acid to generate an enriched nucleic acid solution; and
   3.) separating the sample into at least two subsamples by distributing the enriched nucleic acid solution to two or more assay chambers within the cartridge.

3. The method of embodiment 2, wherein amplifying at least three different target nucleic acid sequences according to step b) comprises performing an amplification reaction within each one of two or more assay chambers in the cartridge while simultaneously detecting amplification product.

4. The method of any one of embodiments 1 to 3, wherein amplifying at least three different target nucleic acid comprises cycling of temperatures, preferably a two-step polymerase chain reaction and a denaturation temperature below 96° C., preferably below 91° C.

5. The method of any one of embodiments 1 to 4, wherein bringing amplification reagents into contact with the subsamples for amplifying at least three different target nucleic acid sequences comprises combining each subsample with at least one dried amplification reagent and at least one dried probe for detection, preferably with at least one dried primer, at least one dried probe and a plurality of dried nucleotides.

6. The method of any one of embodiments 1 to 5, wherein determining the presence or absence of amplification products of the variant target sequence comprises comparing the presence or absence of the amplification product of the variant target sequence to the presence or absence of the amplification product of the identification nucleic acid sequence of the same pathogen.

7. The method of any one of embodiments 1 to 6, wherein the variant target sequence comprises an rRNA sequence or an rRNA encoding sequence.

8. The method of any one of embodiments 1 to 7, wherein separating the sample into at least two subsamples comprises separating the sample into at least 3, at least 4, or at least 5 subsamples.

9. The method of any one of embodiments 1 to 8, wherein at least 1, at least 2, at least 3, at least 4, at least 5, at least 6, at least 7, at least 8, at least 9 or at least 10 additional target nucleic acid sequence(s) is/are determined.

10. The method of embodiment 9, wherein the additional target nucleic acid sequence(s) comprise(s) at least one variant control nucleic acid sequence that is amplified in b).

11. The method of embodiment 9 or 10, wherein the additional target nucleic acid sequence(s) comprise(s) at least one confirmation identification nucleic acid sequence that is amplified in b), wherein each confirmation identification nucleic acid sequence is a sequence comprised in the same pathogen as at least one identification nucleic acid sequence.

12. The method of any one of embodiments 9 to 11, wherein the additional target nucleic acid sequence(s) comprise(s) at least 1, or at least 2 further variant target sequence(s).

13. The method of any one of embodiments 1 to 12, wherein the sample comprises a urine sample, a vaginal swab, a urethral swab, a cervical swab, an anal swab, a rectal swab and/or a pharyngeal swab.

14. The method of any one of embodiments 1 to 13, wherein the pathogens are STIs pathogens.

15. The method of any one of embodiments 1 to 14, wherein the pathogens are bacterial pathogens.

16. The method of embodiment 15, wherein the variant target sequence(s) comprise(s) an antibiotic susceptibility variant target sequence.

17. The method of any one of embodiments 14 to 16, wherein the pathogens comprise or consist of Chlamydia trachomatis and Neisseria gonorrhoeae.

18. The method of embodiment 17, wherein at least one subgroup of the pathogen comprises LGV-causing Chlamydia trachomatis.

19. The method of any one of embodiments 1 to 18, wherein a) the amplification reagents comprise at least; and/or b) determining the presence of amplification products, involves binding of at least:
1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 sequences selected from the group of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.

20. The method of embodiment 19, wherein the amplification reagents comprise at least 1, 2, 3, 4, 5, 6, 7, 8, 9, 10, 11, 12, 13, 14, 15, 16, 17 or 18 sequences selected from the group of: SEQ ID NO: 1, SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 14, SEQ ID NO: 15, SEQ ID NO: 18, SEQ ID NO: 19, SEQ ID NO: 22, SEQ ID NO: 23, SEQ ID NO: 25, SEQ ID NO: 26, SEQ ID NO: 28 and SEQ ID NO: 29.

21. The method of any one of embodiments 18 to 20, wherein
a) at least one identification nucleic acid sequence and at least one identity specifying target sequence are amplified in the same subsample; and/or
b) at least two variant target sequences are amplified in the same subsample.

22. The method of embodiment 21, wherein
a) at least two identification nucleic acid sequences and at least one identity specifying target sequence are amplified in the same subsample; and/or
b) at least one identification nucleic acid sequence and at least two identity specifying target sequences are amplified in the same subsample.

23. The method of any one of embodiments 20 to 22, wherein the amplification reagents comprise at least two or at least three primer pairs
a) selected from the group consisting of:
   i) SEQ ID NO: 1 and SEQ ID NO: 2;
   ii) SEQ ID NO: 25 and SEQ ID NO: 26; and
   iii) SEQ ID NO: 28 and SEQ ID NO: 29;
wherein the at least two or at least three primer pairs are present in the same subsample; and/or
b) selected from the group consisting of:
   i) SEQ ID NO: 4 and SEQ ID NO: 5
   ii) SEQ ID NO: 22 and SEQ ID NO: 23
   iii) SEQ ID NO: 7 and SEQ ID NO: 8,
wherein the at least two or at least three primer pairs are present in the same subsample.

24. The method of any one of embodiments 19 to 23, wherein determining the presence of amplification products, involves presence of
a) SEQ ID NO: 12 and SEQ ID NO: 13 in the same subsample;
b) SEQ ID NO: 16 and SEQ ID NO: 17 in the same subsample; and/or
c) SEQ ID NO: 20 and SEQ ID NO: 21 or SEQ ID NO: 34 and SEQ ID NO: 21 in the same subsample.

25. The method of any one of embodiments 1 to 24, wherein determining the presence of amplification products comprises an optical measurement, preferably an optical measurement of a fluorescent labeled probe.

26. The method of embodiment 25, wherein the optical measurement comprises color compensation filtering.

27. A cartridge comprising sample port assembly, two or more assay chambers and the reagents for performing steps a) to c) of the method of embodiments 2 to 26.

28. A kit comprising the cartridge of embodiment 27 and a cartridge reader for determining whether a sample contains a pathogen according to the method of embodiments 2 to 26.

29. A method for diagnosing a subject with an infectious disease induced by a pathogen variant and/or an infection with a pathogen subgroup, the method comprising the steps of:
(I) determining whether a sample of a subject contains a pathogen according to the method of any one of embodiments 1 to 26;
(II) comparing the determination of step (I) with a diagnosis reference value,
(III) determining based on the comparison in step (II) whether the sample is indicative of:
   a) i) the presence of a first pathogen and ii) a variant and/or subgroup thereof; and/or
   b) i) the presence of a second pathogen and ii) a variant and/or subgroup thereof; and
(IV) diagnosing the subject with an infectious disease induced by at least one pathogen variant and/or an infectious disease induced by at least one pathogen subgroup of at least one pathogen based on the presence in the sample determined in step (III).

30. A method for determining susceptibility of a subject to the treatment with an anti-infective agent, the method comprising the steps of:
(I) determining whether a sample of a subject contains a pathogen according to the method of any one of embodiments 1 to 26;
(II) comparing the determination of step (I) with a susceptibility reference value,
(III) determining based on the comparison in step (II) whether the sample is indicative of the presence a pathogen susceptible to the anti-infective agent; and
(IV) determining susceptibility of the subject to an anti-infective treatment based on the presence in the sample determined in step (III).

31. A pharmaceutical composition comprising an anti-infective agent for use in a subject determined to be susceptible according to the method of embodiment 30.

32. A method of treatment of an infection, the method comprising the step of:
administering to the subject to be treated an effective dose of a pharmaceutical composition, wherein the subject to be treated is
   a) a subject from which a sample is determined to contain a pathogen according to the method of any one of embodiments 1 to 26 and wherein the pharmaceutical composition comprises an active therapeutic agent having known activity against the pathogen(s) present in the sample; and/or
   b) a subject which is determined to be susceptible to the treatment with an anti-infective agent and wherein the pharmaceutical composition comprises the anti-infective agent.

33. The pharmaceutical composition for use in embodiment 31 or the method of treatment of embodiment 32, wherein the pharmaceutical composition comprises at least one therapeutic agent selected from the group consisting of: antibiotic agent, antifungal agent, antiviral agent and anthelmintic agent.

34. A composition comprising reagents for the detection of a pathogen, the composition comprising 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleic acid molecule(s) having the sequence(s) selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.

Accordingly, in one embodiment, the invention relates to a method for determining whether a sample contains a pathogen, the method comprising the steps of: a) separating the sample into at least two subsamples; b) bringing different amplification reagents into contact with at least two of the subsamples for amplifying at least three different target nucleic acid sequences, wherein at least two of the target nucleic acid sequences are identification nucleic acid sequences, wherein each of the identification nucleic acid sequences is characteristic for a pathogen; and 1.) at least one of the target nucleic acid sequences is a variant target sequence, wherein the variant target sequence is indicative of at least one mutation of at least one of the pathogens; and/or 2.) at least one of the target nucleic acid sequences is an identity specifying target sequence, wherein at least one of the identification nucleic acid sequences is characteristic for a plurality of subgroups of at least one of the pathogens and the identity specifying target sequence is comprised in one of the subgroups of the pathogen; c) determining the presence or absence of amplification products of the target nucleic acid sequences in the subsamples using at least one detection probe; and d) determining whether the sample contains a pathogen, wherein presence of an amplification product is an indication of a presence, an absence and/or a quantity of one or more pathogens in the sample.

In certain embodiments, the invention relates to a method for detecting the presence of at least two pathogens in a sample, the method comprising the steps of: a) separating the sample into at least two subsamples; b) amplifying at least three different target nucleic acid sequences in the subsamples, wherein i) at least one target nucleic acid sequence is amplified in each subsample; and ii) at least two of the target nucleic acid sequences are identification nucleic acid sequences, wherein each of the identification nucleic acid sequences is characteristic for a pathogen; and 1.) at least one of the target nucleic acid sequences is a variant target sequence, wherein the variant target sequence is indicative of a mutation of at least one of the pathogens; and/or 2.) at least one of the target nucleic acid sequences is an identity specifying target sequence, wherein at least one of the identification nucleic acid sequences is characteristic for a plurality of subgroups of a pathogen and the identity specifying target sequence is comprised in a subgroup of at least one of the pathogens; c) determining the presence of amplification products of the target nucleic acid sequences in the subsamples; and d) detecting at least two pathogens in a sample based on the amplification products determined in c), wherein presence of an amplification product is an indication of a presence, an absence and/or a quantity of one or more pathogens in the sample.

The term “pathogen”, as used herein, refers to a biological agent that may cause an infection or infectious disease in a host. In some embodiments, the pathogens are at least one selected from the group consisting of virus, bacterium, fungus or parasite. In some embodiments, at least one pathogen is from a certain order, family, genus or species. In some embodiments at least one pathogen is a pathogen with a certain property (e.g. treatment susceptibility) and/or ability (e.g. inducing certain symptoms). Any nucleic acid comprising stadium of a pathogen (e.g. inside the cell or in the virus envelope) may be determined by the method of invention.

The term “sample”, as used herein, refers to any potentially polynucleotide-containing material. In some embodiments, the sample described herein is a sample obtained from a human subject. In some embodiments, the sample described herein is at least one selected from the group consisting of bronchoalveolar lavage, bronchial wash, pharyngeal exudate, tracheal aspirate, blood, serum, plasma, swab, bone, skin, soft tissue, intestinal tract specimen, genital tract specimen, breast milk, lymph, cerebrospinal fluid, pleural fluid, sputum, urine, nasal secretion, tears, bile, ascites fluid, pus, menstrual blood, synovial fluid, vitreous fluid, vaginal secretion, semen and urethral tissue.

The term “subsample”, as used herein, refers to one part of a divided sample. In some embodiments at least two subsamples are of about equal volume. The subsample may be obtained by division of a processed sample.

The term “probe”, as used herein, refers to an oligo that carries at least one label that is used to generate a detectable signal. Probes typically have a blocked 3′ end to prevent their extension by enzymes with polymerase activity. In certain embodiments, the number of detection probes is at least a third, at least half, at least two thirds or equal the number of target nucleic acid sequences.

By bringing different amplification reagents into contact with at least two of the subsamples, at least three different target nucleic acid sequences can be amplified in the subsamples. Typically, the amplification reagents are chosen such that a different target nucleic acid sequence is amplified in each subsample. However, duplications, negative controls may also be present.

Amplification of the nucleic acid sequence can be achieved by any method known in the art (see e.g. Monis, P. T., & Giglio, S., 2006, Infection, Genetics and Evolution, 6 (1), 2-12.) In some embodiments, the amplification technique is selected from the group consisting of PCR, ligation-mediated amplification, transcription-based amplification, TMA, NASBA, LCR, linear linked amplification and SDA. Amplification products include products from direct amplification of the target nucleic acid sequence and/or subsequent products formed therefrom.

The term “target nucleic acid sequence”, as used herein, refers to a nucleic acid sequence, preferably an RNA or DNA sequence, where the skilled person is aware that it may be comprised in a sample. In particular, the target nucleic acid sequence, within the present invention, is a sequence that is detectable using the method of the present invention. That is, a nucleic acid sequence available to the skilled person is a target nucleic acid sequence if the skilled person can determine whether the sequence will likely be detectable in a sample with the methods as provided herein. Within the present invention, the target nucleic acid sequence comprises at least one primer binding site.

Determination of the presence of amplification products of sequences can be achieved by any method known in the art. However, it is preferred that the presence of amplification products such as double-stranded DNA amplification products is determined by using a nucleic acid molecule hybridisable to the amplification product, in particular wherein the nucleic acid molecule is labelled, using a molecule that intercalates in the amplification products or using turbidity measurement. The term “label” or grammatical variations thereof, as used herein, refer to any detectable or signal-generating molecule or reporter molecule. Convenient labels include colorimetric, chemiluminescent, chromogenic, radioactive and fluorescent labels, but enzymatic (e.g. colorimetric, luminescent, chromogenic) or antibody-based labelling methods or signal-generating systems may also be used. Thus, the term “label” as used herein includes not only directly detectable signal-giving or passive moieties, but also any moiety which generates a signal or takes part in a signal generating reaction or that may be detected indirectly in some way. “labelled” as used herein, refers to being connected with or linked to a detectable label.

Determining whether an amplification product is present in the sample may be achieved via fluorescence reporting. As used herein, an agent or dye that “intercalates” refers to an agent or moiety capable of non-covalent insertion between stacked base pairs in a nucleic acid double helix.

The term “identification nucleic acid sequence”, as used herein, refers to a sequence that is characteristic for a pathogen order, family, genus and/or species. A sequence is characteristic for a pathogen if it can be found in most pathogens in the group of interest, preferably all relevant pathogens in the group of interest, more preferably all pathogens in the group of interest (e.g., pathogen order group, pathogen family group, pathogen genus group, pathogen species group). For example, an identification nucleic acid sequence is characteristic for Chlamydia trachomatis if the identification nucleic acid sequence can be found in most Chlamydia trachomatis, preferably all relevant Chlamydia trachomatis (i.e., in all clinically relevant Chlamydia trachomatis), more preferably all Chlamydia trachomatis.

In some embodiments, the identification nucleic acid sequence is specific for the pathogens in the group of interest in that it is not found in any other relevant organism, preferably it is not found in any other organism. As such the specificity of the method at least partially depends on how specific the identification nucleic acid sequence is for the pathogens in the group of interest.

The person skilled in the art is aware of which are relevant pathogens or relevant organisms for which cross-reactivity must be avoided to minimize false positives. Such relevant pathogens or relevant organisms can be pathogens or organisms that are closely related, show a different pathology, a different treatment susceptibility and/or often co-occur in the source of the sample. For example, an identification nucleic acid sequence is specific for Chlamydia trachomatis, if the nucleic acid sequence is not found in other organisms and cells that are typically found in urine samples, vaginal swabs, urethral swabs, cervical swabs, anal swabs, rectal swabs and/or pharyngeal swabs.

The term “variant target sequence” refers to a target nucleic acid sequence that is indicative of a variant of a pathogen with an altered clinically relevant property compared to another variant of the same pathogen (such as a WT pathogen) with a reference sequence (e.g. WT sequence). In some embodiments, the variant target sequence described herein is a target sequence indicative of a variant having at least one altered clinically relevant property selected from the group of pathogenicity, transmissibility and drug susceptibility. Two variant target sequences described herein can be distinguished from each other without the use of a sequencing method by a different binding of the probe and/or primer. As such the variant target sequence comprises at least one mutation compared to a reference sequence. The mutation can be a replacement, deletion or insertion. Typically, the mutation of the variant target sequence extends over between 1 and 100, 1 and 75, 1 and 50, 1 and 25, 1 and 20, 1 and 10, 1 and 9, 1 and 8, 1 and 7, 1 and 6, 1 and 5, 1 and 4, 1 and 3 or 1 and 2 nucleotides and remains distinguishable by primer and/or probes of a non-sequencing method. In some embodiments, distinguishing of the variant target sequence from a reference sequence is enabled by the binding of the probe. Therefore, whether the probe binds to the amplification product of a variant target sequence is indicative of whether the target sequence belongs to a variant or a reference (e.g. WT) thereof. In some embodiments, distinguishing of the variant target sequence from a reference sequence is enabled by the binding of the primer. Therefore, whether the probe binds to the amplification product of a variant target sequence is indicative of whether the target sequence belongs to a variant or a reference (e.g. WT) thereof.

In some embodiments, the mutation is a single-nucleotide replacement, deletion or insertion compared to a reference sequence, wherein the difference in the single nucleotide influences at least one property and/or ability of the pathogen. In some embodiments, the reference nucleic acid sequence described herein is a wild type sequence of the same pathogen.

The absence of one or more pathogens in the sample may be determined, e.g., by an internal control. The quantity of one or more pathogens in the sample may be determined, e.g., based on a Ct-value and/or a reference measurement. In some embodiments, the quantity described herein is a relative quantity. In some embodiments, the quantity described herein is an absolute quantity.

The term “identity specifying target sequence”, as used herein, refers to a sequence that is found in a subgroup of the order, family, genus and/or species identifiable by at least one of the identification nucleic acid sequences. As such, the identity specifying target sequence is a predetermined sequence. For example, while the identification nucleic acid sequence may identify Chlamydia trachomatis, the identity specifying target sequence may identify lymphogranuloma venereum (LGV) inducing subgroups of Chlamydia trachomatis. In certain embodiments, the skilled person uses a library-based approach to identify subspecies specific target sequences, e.g., as described in the examples section.

The inventors found that with the means and methods described herein faster and more accurate tests to identify at least two pathogens and at least one characteristic thereof can be achieved. The method may also determine whether only one of the pathogens is present. By using the means and methods of the invention results can be provided while the patient is still available for accurate treatment. Such an early intervention is highly relevant in many diseases induced by pathogens and improves treatment choice, treatment compliance and disease outcome.

Accordingly, the invention is at least in part based on the finding that fast and accurate detection of pathogens and pathogen characteristics as described herein are possible.

In certain embodiments, the invention relates to a method for detecting the presence of at least two pathogens in a sample, the method comprising the steps of: a) separating the sample into at least two subsamples; b) amplifying at least three different target nucleic acid sequences in the subsamples, wherein i) at least one target nucleic acid sequence is amplified in each subsample; and ii) at least two of the target nucleic acid sequences are identification nucleic acid sequences, wherein each of the identification nucleic acid sequences is characteristic for a pathogen; and at least one of the target nucleic acid sequences is a variant target sequence, wherein the variant target sequence is indicative of a mutation of at least one of the pathogens; c) determining the presence of amplification products of the target nucleic acid sequences in the subsamples; and d) detecting at least two pathogens in a sample based on the amplification products determined in c), wherein presence of an amplification product is an indication of a presence, an absence and/or a quantity of one or more pathogens in the sample.

In certain embodiments, the invention relates to a method for determining whether a sample contains a pathogen, the method comprising the steps of: a) separating the sample into at least two subsamples; b) bringing different amplification reagents into contact with at least two of the subsamples for amplifying at least four different target nucleic acid sequences, wherein at least two of the target nucleic acid sequences are identification nucleic acid sequences, wherein each of the identification nucleic acid sequences is characteristic for a pathogen; and 1.) at least one of the target nucleic acid sequences is a variant target sequence, wherein the variant target sequence is indicative of at least one mutation of at least one of the pathogens; and 2.) at least one of the target nucleic acid sequences is an identity specifying target sequence, wherein at least one of the identification nucleic acid sequences is characteristic for a plurality of subgroups of at least one of the pathogens and the identity specifying target sequence is comprised in one of the subgroups of the pathogen; c) determining the presence or absence of amplification products of the target nucleic acid sequences in the subsamples using at least one detection probe; and d) determining whether the sample contains a pathogen, wherein presence of an amplification product is an indication of a presence, an absence and/or a quantity of one or more pathogens in the sample.

In certain embodiments, the invention relates to a method for detecting the presence of at least two pathogens in a sample, the method comprising the steps of: a) separating the sample into at least two subsamples; b) amplifying at least three different target nucleic acid sequences in the subsamples, wherein i) at least one target nucleic acid sequence is amplified in each subsample; and ii) at least two of the target nucleic acid sequences are identification nucleic acid sequences, wherein each of the identification nucleic acid sequences is characteristic for a pathogen; and at least one of the target nucleic acid sequences is an identity specifying target sequence, wherein at least one of the identification nucleic acid sequences is characteristic for a plurality of subgroups of a pathogen and the identity specifying target sequence is comprised in a subgroup of at least one of the pathogens; c) determining the presence of amplification products of the target nucleic acid sequences in the subsamples; and d) detecting at least two pathogens in a sample based on the amplification products determined in c), wherein presence of an amplification product is an indication of a presence, an absence and/or a quantity of one or more pathogens in the sample.

In certain embodiments, the invention relates to the method of the invention, wherein separating the sample according to step a) comprises the substeps of: 1.) accepting a cartridge having a sample port assembly containing the sample; 2.) enriching the nucleic acid to generate an enriched nucleic acid solution; and 3.) separating the sample into at least two subsamples by distributing the enriched nucleic acid solution to two or more assay chambers within the cartridge.

The term “cartridge”, as used herein, refers to a reagent pack to deliver individual reagents or consumables (fluid and solid), each contained within a compartment within the cartridge and each reagent sealed so as to prevent contamination from one compartment to another compartment by reagent aspiration, spill over and so on. Use of a cartridge with a plurality of sealed compartments permits delivery or acceptance of a fluid or solid while isolating the contents of the cartridge from the external environment and protecting the user from exposure to the fluid contents.

The use of a cartridge allows the method of the invention to be used outside of specialized laboratories and may simplify its use. This enables on-site testing, reduces the need for trained personal and accelerates the detection procedure.

Accordingly, the invention is at least in part based on the finding that fast and accurate detection of pathogens and pathogen characteristics by using a cartridge as described herein is possible.

In certain embodiments, the invention relates to the method of the invention, wherein amplifying at least three different target nucleic acid sequences according to step b) comprises performing an amplification reaction within each one of two or more assay chambers in the cartridge while simultaneously detecting amplification product.

The phrase “simultaneously detecting”, in the context of an amplification product, refers to a detection process that at least overlaps with the amplification reaction.

The amplification reaction and the simultaneous detection process might be achieved in at least two assay chambers at the same time, one chamber after the other, or alternating between the chambers.

Accordingly, the invention is at least in part based on the finding that simultaneous detection can enhance accuracy and reduce testing time as described herein.

In certain embodiments, the invention relates to the method of the invention, wherein amplifying at least three different target nucleic acid comprises cycling of temperatures.

Cycling of temperatures may be achieved by heating and cooling a chamber or by moving the sample, subsample and/or reagent alternating into colder and warmer chambers.

Typically, cycling of temperatures serves the purpose of accomplishing a denaturing step, an annealing step, and an extension step in a nucleic acid amplification reaction.

In certain embodiments, the invention relates to the method of the invention, wherein amplifying at least three different target nucleic acid comprises a two-step polymerase chain reaction and a denaturation temperature below 96° C.

In certain embodiments, the invention relates to the method of the invention, wherein amplifying at least three different target nucleic acid comprises a two-step polymerase chain reaction with a denaturation temperature below 91° C.

The inventors found that a reduced denaturation temperature can reduce the heating time and accelerate the detection method.

Accordingly, the invention is at least in part based on the finding that the method is accelerated by a reduced denaturation temperature.

In certain embodiments, the invention relates to the method of the invention, wherein amplifying at least three different target nucleic acid sequences comprises combining each subsample with at least one dried amplification reagent.

The term “dried amplification reagent”, as used herein, refers to a solid amplification reagent that is free of water or almost free of water (may comprise e.g. water of crystallization) and/or a reagent wherein water and/or other solvents have been removed by a drying method such as freeze drying.

In some embodiments, the invention relates to the method of the invention, wherein the probe is dried.

In certain embodiments, the invention relates to the method of the invention, wherein amplifying at least three different target nucleic acid sequences comprises combining each subsample with at least one dried primer a plurality of dried nucleotides and at least one dried probe for detection.

The term “primer”, as used herein, refers to a nucleic acid molecule comprising a 3′-terminal —OH group that, upon hybridization to a complementary nucleic acid sequence, can be elongated, e.g., via an enzymatic nucleic acid replication reaction. Within the present invention, the target nucleic acid sequence comprises at least one primer binding site that is at least partially identical to at least one of the primers used in the methods of the invention. Primer binding sites are considered identical to a primer site if the sequence is exactly identical or if they differ only in that one sequence comprises uracil instead of thymidine and/or if they differ only in that one sequence comprises one or more modified nucleotides instead of the respective non-modified nucleotide(s). Some primers that can be used in the method of the invention are exemplified herein. However, the skilled person is well-aware how to design alternative or further primer sequences depending on the target sequence to be detected in the sample (see e.g., Bustin, Stephen, and Jim Huggett, 2017, Biomolecular detection and quantification 14:19-28; Jia, B., et al., 2019, Frontiers in microbiology, 10, 2860).

The use of dried reagents allows the cartridge to be stored and used at room temperature.

Accordingly, the invention is at least in part based on the finding that the reagents for the method of the invention have a prolonged shelf life and stability.

In certain embodiments, the invention relates to the method of the invention, wherein determining the presence of amplification products of the variant target sequence comprises comparing the presence of the amplification product of the variant target sequence to the presence of the amplification product of the identification nucleic acid sequence of the same pathogen.

Some variant target sequences (e.g. sequences comprising SNPs) can be found on more than one species, e.g., additionally on a species that is not intended to be detected by the method of the invention. By the comparison of the amplification product of the variant target sequence to the presence of the amplification product of the identification nucleic acid sequence of the same pathogen, false positives can be excluded.

Accordingly, the invention is at least in part based on the finding that the comparison as described herein reduces the number of false positives.

In certain embodiments, the invention relates to the method of the invention, wherein the variant target sequence comprises an rRNA sequence or an rRNA encoding sequence.

The inventors found that rRNA and the encoding sequence thereof (e.g. the DNA encoding the rRNA) comprises particularly useful information in the context of the invention.

Accordingly, the invention is at least in part based on the finding that rRNA-associated information enhances the accuracy of the method described herein.

In certain embodiments, the invention relates to the method of the invention, wherein separating the sample into at least two subsamples comprises separating the sample into at least 3 subsamples.

In certain embodiments, the invention relates to the method of the invention, wherein separating the sample into at least two subsamples comprises separating the sample into at least 4 subsamples.

In certain embodiments, the invention relates to the method of the invention, wherein separating the sample into at least two subsamples comprises separating the sample into at least 5 subsamples.

The inventors found, that separating the sample into several subsamples increases the number of pathogens and/or properties of pathogens that can be accurately detected in a short period of time, e.g. simultaneously.

In certain embodiments, the invention relates to the method of the invention, wherein separating the sample into at least two subsamples comprises separating the sample into between 2 to 20, between 3 to 15, between 4 to 10, between 4 and 7, or 5 subsamples.

The inventors found that separating the sample into too many subsamples increases the required sample volume or reduces the number of nucleic acid molecules per subsample. Therefore, a certain range of number of subsamples is particularly useful in the context of the invention. The optimal number of subsamples depends amongst others on the (number of) target pathogens, (number of) target sequences, detection method and potential contaminants.

Accordingly, the invention is at least in part based on the finding that a certain number of subsamples is particularly beneficial for efficient detection as described herein.

In certain embodiments, the invention relates to the method of the invention, wherein at least 1, at least 2, at least 3, at least 4, at least 5, at least 6, at least 7, at least 8, at least 9 or at least 10 additional target nucleic acid sequence(s) is/are determined.

In certain embodiments, the invention relates to the method of the invention, wherein at least 2 target nucleic acid sequences are determined in each subsample.

In certain embodiments, the invention relates to the method of the invention, wherein 2 or 3 target nucleic acid sequences are determined in each subsample.

The inventors found that determining several target nucleic acid sequences increases accuracy and/or efficiency (reduced required volume, reduced time) but determining and/or detecting too many target nucleic acid sequences may result in artefacts.

Accordingly, the invention is at least in part based on the accuracy and/or efficiency resulting from determining a certain number of nucleic acid sequences in a certain number of subsamples.

In certain embodiments, the invention relates to the method of the invention, wherein the additional target nucleic acid sequence(s) comprise(s) at least one variant control nucleic acid sequence that is amplified in b).

The term “variant control nucleic acid sequence”, as used herein, refers to a sequence that is absent in a pathogen having the variant target sequence. In one example the variant control nucleic acid sequence described herein is a wild type sequence and the variant target sequence a sequence with a mutation, e.g. a SNP.

By the use of a variant control nucleic acid sequence, false positive and/or false negative interpretation of amplification products of the variant target sequence can be excluded, and accuracy of the method can be improved.

Accordingly, the invention is at least in part based on the finding that a control sequence can improve the accuracy of the method as described herein.

In certain embodiments, the invention relates to the method of the invention, wherein the additional target nucleic acid sequences comprise at least one variant target sequence and at least one variant control nucleic acid sequence thereof.

Preferably, the variant target sequence and the variant control nucleic acid sequence thereof are amplified by the same primer(s) and the detection is achieved by a probe that is specific for the variant target sequence or the variant control nucleic acid sequence thereof, respectively.

In certain embodiments, the invention relates to the method of the invention, wherein the additional target nucleic acid sequence(s) comprise(s) at least one confirmation identification nucleic acid sequence that is amplified in b), wherein each confirmation identification nucleic acid sequence is a sequence comprised in the same pathogen as at least one identification nucleic acid sequence.

In certain embodiments, the invention relates to the method of the invention, wherein the additional target nucleic acid sequence(s) comprise(s) at least 1, or at least 2 further variant target sequence(s).

The inventors found that detection of (a) further identification nucleic acid sequence(s) and/or (an) additional target nucleic acid sequence(s) enables fast and efficient additional characterization of the identity and/or property/properties of the pathogens of interest.

In certain embodiments, the invention relates to the method of the invention, wherein the sample comprises a urine sample, a vaginal swab, a urethral swab, a cervical swab, an anal swab, a rectal swab and/or a pharyngeal swab.

The term “swab”, as used herein, refers to a sample obtained by a swabbing method. As such the swab is usually processed, e.g., by extraction into a buffer. The swab described herein may be a single swab or a pooled swab. The pooled swab is typically a sample comprising several swabs from the same person from the same or different sampling sites. The swabs can be taken by any swabbing method known in the art and the sampling sites may be interpreted by the person skilled in the art using common knowledge. For example, the cervical swab described herein may also include endocervical swabs.

In certain embodiments, the invention relates to the method of the invention, wherein the sample is a sample from a subject with a history of sexual activity, preferably a history of sexual activity with an above average number of sexual partners and/or a history of sexual activity with a partner with an above average number of sexual partners. In certain embodiments, the invention relates to the method of the invention, wherein the sample is a sample from a subject in the reproductive age. The term “subject”, as used herein, refers to a mammal, preferably a human. In certain embodiments, the invention relates to the method of the invention, wherein the sample is a sample from an MSM (men who have sex with men) subject. In some embodiments, the invention relates to the method of the invention, wherein the sample is a triple swab (e.g., anal/rectal, urethral and pharyngeal swab).

The fast and minimally invasive specimen acquisition is of importance for facilitating the use and efficiency of the method of the invention, in particular in vulnerable patient populations. Urine samples, vaginal swabs, urethral swabs, cervical swabs, rectal swabs and/or pharyngeal swabs can be obtained without the need for specialized infrastructure.

Accordingly, the invention is at least in part based on the finding that certain sample types are particularly useful for the method of the invention.

In certain embodiments, the invention relates to the method of the invention, wherein at least one of the pathogens is an STIs pathogen.

The term “STI pathogen”, as used herein, refers to a pathogen that is sexually transmissible. In some embodiments, the STI pathogen is at least one selected from the group consisting of mycobacterium genitalium, HIV, chlamydia, gonorrhea, trichomoniasis, genital warts, genital herpes, pubic lice, scabies, syphilis and human papillomavirus.

In certain embodiments, the invention relates to the method of the invention, wherein the pathogens are bacterial pathogens.

In some embodiments, the invention relates to the method of the invention, wherein the pathogens comprise at least one bacteria from the genus selected from the group consisting of Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus AlteromonasAmycolata, Amycolatopsis, Anaerobospirillum, Anaerorhabdus, “Anguillina”, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Flexispira, Francisella, Fusobacterium, Gardnerella, Gemella Globicatella, Gordona, Haemophilus, Hafnia, Helicobacter, Helococcus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Plesiomonas Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia and Yokenella.

In some embodiments, the method of the invention is used for detecting the presence of a nucleic acid sequence of at least one virus of the genus selected from the group consisting of Adenoviridae, Anelloviridae, Arenaviridae, Astroviridae, Bunyaviridae, Bunyavirus, Caliciviridae, Coronaviridae, Filoviridae, Flaviviridae, Hepadnaviridae, Herpesviridae, Orthomyxoviridae, Papillomaviridae, Paramyxoviridae, Parvoviridae, Picornaviridae, Pneumoviridae, Polyomaviridae, Poxviridae, Reoviridae, Retroviridae, Rhabdoviridae, Rhabdovirus, Togaviridae.

In some embodiments, the method of the invention is used for detecting the presence of a nucleic acid sequence of at least one fungus of the genus selected from the group consisting of Candida, Aspergillus, Cryptococcus, Histoplasma, Pneumocystis and/or Stachybotrys.

In some embodiments, the method of the invention is used for detecting the presence of a nucleic acid sequence of at least one parasite from the genus selected from the group consisting of ectoparasites, protozoan organisms and/or helminths such as tapeworms, flukes and/or roundworms.

In certain embodiments, the invention relates to the method of the invention, wherein the variant target sequence is an antibiotic susceptibility variant target sequence.

The term “antibiotic susceptibility variant target sequence”, as used herein, refers to an variant target sequence that is associated with antibiotic susceptibility of the pathogen. Such variant sequences, e.g. SNPs, are well known and documented in the art (see e.g., Alcock et al, 2020, Nucleic Acids Research, 48, D517-D525).

The knowledge of antibiotic susceptibility of a pathogen is central for an adequate treatment choice.

Accordingly, the invention is at least in part based on the finding that detecting antibiotic susceptibility variant target sequences can improve treatment decisions.

In certain embodiments, the invention relates to the method of the invention, wherein the pathogens comprise or consist of Chlamydia trachomatis and Neisseria gonorrhoeae.

In certain embodiments, the invention relates to the method of the invention, wherein the pathogens comprise or consist of Chlamydia trachomatis and Neisseria gonorrhoeae and at least one subgroup of Chlamydia trachomatis and/or Neisseria gonorrhoeae.

In certain embodiments, the invention relates to the method of the invention, wherein the subgroups of the pathogen comprise LGV-causing Chlamydia trachomatis.

The term “LGV-causing Chlamydia trachomatis”, as used herein, refers to strains of Chlamydia trachomatis causing LGV. In some embodiments, the LGV-causing Chlamydia trachomatis is at least one selected from the group consisting of: Chlamydia trachomatis serovars L1, Chlamydia trachomatis serovars L2 and Chlamydia trachomatis serovars L3.

The rapid identification of LGV-causing Chlamydia trachomatis can have a direct impact on treatment decision, which impacts the opportunity to catch the more dangerous variants of Chlamydia trachomatis early on before it is further spread and minimizes unnecessary treatment of the less harmful Chlamydia trachomatis variants.

In certain embodiments, the invention relates to the method of the invention, wherein amplifying at least three different target nucleic acid sequences and/or determining the presence of amplification products, involves binding of at least 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30 sequence(s) selected from the group of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.

The inventors found that means and methods described herein, can at least be partially improved by replacing SEQ ID NO: 20 with SEQ ID NO: 34.

In certain embodiments, the invention relates to the method of the invention, wherein amplifying at least three different target nucleic acid sequences involves binding of at least 1, 2, 3, 4, 5, 6, 7, 8, 9, 10, 11, 12, 13, 14, 15, 16, 17 or 18 sequences selected from the group of: SEQ ID NO: 1, SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 14, SEQ ID NO: 15, SEQ ID NO: 18, SEQ ID NO: 19, SEQ ID NO: 22, SEQ ID NO: 23, SEQ ID NO: 25, SEQ ID NO: 26, SEQ ID NO: 28 and SEQ ID NO: 29.

In certain embodiments, the invention relates to the method of the invention, wherein at least one identification nucleic acid sequence and at least one identity specifying target sequence are amplified in the same subsample.

In certain embodiments, the invention relates to the method of the invention, wherein at least two variant target sequences are amplified in the same subsample.

In certain embodiments, the invention relates to the method of the invention, wherein a) at least one identification nucleic acid sequence and at least one identity specifying target sequence are amplified in the same subsample; and b) at least two variant target sequences are amplified in the same subsample.

In certain embodiments, the invention relates to the method of the invention, wherein at least two identification nucleic acid sequences and at least one identity specifying target sequence are amplified in the same subsample.

In certain embodiments, the invention relates to the method of the invention, wherein at least one identification nucleic acid sequence and at least two identity specifying target sequences are amplified in the same subsample.

In certain embodiments, the invention relates to the method of the invention, wherein a) at least two identification nucleic acid sequences and at least one identity specifying target sequence are amplified in the same subsample; and b) at least one identification nucleic acid sequence and at least two identity specifying target sequences are amplified in the same subsample.

In certain embodiments, the invention relates to the method of the invention, wherein at least two variant target sequences are amplified in the same subsample.

In certain embodiments, the invention relates to the method of the invention, wherein a) at least one identification nucleic acid sequence and at least one identity specifying target sequence are amplified in the same subsample; and b) at least two variant target sequences are amplified in the same subsample.

In certain embodiments, the invention relates to the method of the invention, wherein a) at least two identification nucleic acid sequences and at least one identity specifying target sequence are amplified in the same subsample; b) at least one identification nucleic acid sequence and at least two identity specifying target sequences are amplified in the same subsample; and c) at least two variant target sequences are amplified in the same subsample.

In some embodiments, the at least one target gene or target region described in table 3 is targeted.

In certain embodiments, the invention relates to the method of the invention, wherein amplifying at least three different target nucleic acid sequences involves the presence of at least two or at least three primer pairs a) selected from the group consisting of: i) SEQ ID NO: 1 and SEQ ID NO: 2; ii) SEQ ID NO: 25 and SEQ ID NO: 26; and iii) SEQ ID NO: 28 and SEQ ID NO: 29; wherein the at least two or at least three primer pairs are present in the same subsample; and/or b) selected from the group consisting of: i) SEQ ID NO: 4 and SEQ ID NO: 5; ii) SEQ ID NO: 22 and SEQ ID NO: 23; and iii) SEQ ID NO: 7 and SEQ ID NO: 8, wherein the at least two or at least three primer pairs are present in the same subsample.

In certain embodiments, the invention relates to the method of the invention, wherein determining the presence of amplification products, involves presence of a) SEQ ID NO: 12 and SEQ ID NO: 13 in the same subsample; b) SEQ ID NO: 16 and SEQ ID NO: 17 in the same subsample; and/or c) SEQ ID NO: 20 and SEQ ID NO: 21 or SEQ ID NO: 34 and SEQ ID NO: 21 in the same subsample.

In certain embodiments, the invention relates to the method of the invention, wherein determining the presence of amplification products, involves presence pairs of probes a) having a sequence as defined by SEQ ID NO: 12 and SEQ ID NO: 13 and being in one subsample; b) having a sequence as defined by SEQ ID NO: 16 and SEQ ID NO: 17 and being in one subsample; and/or c) having a sequence as defined by SEQ ID NO: 20 and SEQ ID NO: 21 or SEQ ID NO: 34 and SEQ ID NO: 21 and being in one subsample.

As such, the two different probes can distinguish the presence or absence of a mutation in an amplification product from a primer pair.

The inventors found that two different probes binding to an amplification product of the same primer pairs is particularly useful to detect variant-derived properties such as antibiotic susceptibility. The inventors found that the oligonucleotides described herein are surprisingly useful for the means and methods described herein.

In some embodiments, the invention relates to the method of the invention, wherein amplifying at least three different target nucleic acid sequences and/or determining the presence of amplification products, involves binding of at least one sequence comprising at least one sequence part that is chemically modified to increase stability, binding selectivity and/or reduction of background noise (e.g. background fluorescence).

Such chemically modified sequence parts are known in the art and include locked nucleic acids and minor groove binder nucleotides.

Minor groove binder molecules are typically conjugated to oligonucleotides such as probes and can form stable duplexes with single-stranded nucleic acid targets.

In some embodiments, the invention relates to the method of the invention, wherein amplifying at least three different target nucleic acid sequences and/or determining the presence of amplification products, involves binding of at least one sequence comprising at least one locked nucleic acid.

The term “locked nucleic acid”, as used herein, refers to a nucleic acid that exhibits reduced the conformational flexibility of the ribose and increased local organization of the phosphate backbone. This is typically achieved by a methylene bridge that connects the 2′-oxygen of ribose with the 4′-carbon, to result in a locked 3′-endo conformation.

Chemically modified sequence parts can increase stability against enzymatic degradation and/or improve specificity and/or affinity of the sequences described herein.

Accordingly, the invention is at least in part based on the finding that chemically modified sequence parts enhance robustness and specificity of the means and methods described herein.

In certain embodiments, the invention relates to the method of the invention, wherein determining the presence of amplification products comprises an optical measurement.

The term “optical measurement”, as used herein, refers to light based measurement comprising an emitter and a sensor. The optical measurement described herein may be light-absorption or fluorescence based.

In certain embodiments, the invention relates to the method of the invention, wherein determining the presence of amplification products comprises measurement of a labeled probe.

The term “label” or grammatical variations thereof, as used herein, refer to any detectable or signal-generating molecule or reporter molecule. Convenient labels include colorimetric, chemiluminescent, chromogenic, radioactive and fluorescent labels, but enzymatic (e.g. colorimetric, luminescent, chromogenic) or antibody-based labelling methods or signal-generating systems may also be used. Thus, the term “label” as used herein includes not only directly detectable signal-giving or passive moieties, but also any moiety which generates a signal or takes part in a signal generating reaction or that may be detected indirectly in some way. “labelled” as used herein, refers to being connected with or linked to a detectable label. In certain embodiments, the invention relates to the method of the invention, wherein determining the presence of amplification products comprises an optical measurement of a fluorescent labeled probe. Determining whether an amplification product is present in the sample may be achieved by a fluorescence technique that relies on the mechanism of Förster resonance energy transfer (FRET) (Chen Q, et al., 1997, Biochemistry 36 (15): 4701-11). The design of fluorescent labeled probes is known in the art (see e.g. Jothikumar, P., Hill, V., & Narayanan, J., 2009, Biotechniques, 46 (7), 519-524)

In certain embodiments, the invention relates to the method of the invention, wherein the optical measurement comprises color compensation filtering.

Color compensation filtering can be used to compensate for overlaps in the spectra of fluorescent labels.

Optical measurement, fluorescent labels and/or color compensation filtering are tools that surprisingly improve the accuracy, time requirements and potential to detect several pathogens and characteristics thereof simultaneously of the means and methods described herein.

In certain embodiments, the invention relates to a cartridge comprising sample port assembly, two or more assay chambers and the reagents for performing steps a) to c) of the method of the invention.

In certain embodiments, the invention relates to a kit comprising the cartridge of the invention and a cartridge reader for detecting the presence of at least two pathogens in a sample according to the method of the invention.

In certain embodiments, the invention relates to a kit comprising the cartridge of the invention and a cartridge reader for determining whether a sample contains a pathogen according to the method of the invention.

In certain embodiments, the invention relates to a method for diagnosing a subject with an infectious disease induced by a pathogen variant and/or an infection with a pathogen subgroup, the method comprising the steps of: (I) determining whether a sample of a subject contains a pathogen according to the method of the invention; (II) comparing the determination of step (I) with a diagnosis reference value, (III) determining based on the comparison in step (II) whether the sample is indicative of: a) i) the presence of a first pathogen and ii) a variant and/or subgroup thereof; and/or b) i) the presence of a second pathogen and ii) a variant and/or subgroup thereof; and (IV) diagnosing the subject with an infectious disease induced by at least one pathogen variant and/or an infectious disease induced by at least one pathogen subgroup of at least one pathogen based on the presence in the sample determined in step (III).

The diagnosis reference value can be any value, matrix or pattern. As such the diagnosis reference value can for example be a Ct threshold, a signal intensity threshold or a detection algorithm as described in the examples section.

In certain embodiments, the invention relates to a method for determining susceptibility of a subject to the treatment with an anti-infective agent, the method comprising the steps of: (I) determining whether a sample of a subject contains a pathogen according to the method of the invention; (II) comparing the determination of step (I) with a susceptibility reference value, (III) determining based on the comparison in step (II) whether the sample is indicative of the presence a pathogen susceptible to the anti-infective agent; and (IV) determining susceptibility of the subject to an anti-infective treatment based on the presence in the sample determined in step (III).

In certain embodiments, the invention relates to a pharmaceutical composition comprising an anti-infective agent for use in a subject determined to be susceptible according to the method of the invention.

In certain embodiments, the invention relates to a method of treatment of an infection, the method comprising the step of: administering to the subject to be treated an effective dose of a pharmaceutical composition, wherein the subject to be treated is a) a subject from which a sample is determined to contain a pathogen according to the method of the invention and wherein the pharmaceutical composition comprises an active therapeutic agent having known activity against the pathogen(s) present in the sample; and/or b) a subject which is determined to be susceptible to the treatment with an anti-infective agent and wherein the pharmaceutical composition comprises the anti-infective agent.

In certain embodiments, the invention relates to the pharmaceutical composition for use in the invention or the method of treatment of the invention, wherein the pharmaceutical composition comprises at least one therapeutic agent selected from the group consisting of: antibiotic agent, antifungal agent, antiviral agent and anthelmintic agent. In some embodiments the therapeutic agent is a treatment compound described herein, preferably a fluoroquinolones (e.g., ciprofloxacin) or macrolides (e.g., azithromycin).

In certain embodiments, the invention relates to a composition comprising reagents for the detection of a pathogen, the composition comprising 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleic acid molecule(s) having the sequence(s) selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.

In certain embodiments, the invention relates to a composition comprising reagents for the detection of a pathogen, the composition comprising 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleic acid molecule(s) having the sequence(s) selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 34, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.

In certain embodiments, the invention relates to a composition comprising reagents for the detection of a pathogen, the composition comprising 1, 2, 3, 4, 5, 6, 7, 8, 9, 10, 11, 12, 13, 14, 15, 16, 17, 18, 19, 20, 21, 22, 23, 24, 25, 26, 27 or 28 nucleic acid molecule(s) having the sequence(s) a) selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30 or b) or a sequence of a), wherein 1 or 2 of the nucleic acids are deleted, inserted or replaced by a different one without (or without substantially) changing the binding site or without (or without substantially) lowering binding affinity to the binding site.

In certain embodiments, the invention relates to a composition comprising reagents for the detection of a pathogen, the composition comprising 1, 2, 3, 4, 5, 6, 7, 8, 9, 10, 11, 12 nucleic acid molecule(s) having a) the sequence(s) selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30 or b) or a sequence of a), wherein 1 or 2 of the nucleic acids are deleted, inserted or replaced by a different one without (or without substantially) changing the binding site or without (or without substantially) lowering binding affinity to the binding site.

In certain embodiments, the invention relates to a method for determining treatment susceptibility, the method comprising the steps of: 1. Detecting the presence of at least two pathogens in a sample of a subject according to the method of the invention; 2. Determining treatment susceptibility of the subject based on the detection of the pathogen(s) in step 1), wherein a subject is only evaluated for susceptibility to a treatment if a pathogen is present.

As such, the pathogen or the pathogen and properties of a pathogen (e.g., antibiotic resistance markers) may be determined and influence the decision, whether a subject is susceptible to a treatment.

For example, a subject may be considered not susceptible to a certain antibiotic treatment in absence of a certain antibiotic susceptibility variant target nucleic acid (e.g., an SNP indicative of antibiotic resistance). It may then be considered susceptible to an alternative antibiotic treatment, to which no resistance has been determined.

In certain embodiments, the invention relates to a method of treatment the method comprising the steps of: 1. detecting the presence of at least two pathogens in a sample of a subject according to the method of the invention; and 2. treating the subject based on the detected presences of the pathogens in step 1), preferably wherein the subject is determined as susceptible according to the method for determining treatment susceptibility described herein.

In certain embodiments, the invention relates to a treatment compound for use in a subject determined susceptible according to the method described herein.

The term “treatment compound”, as used herein, refers to any compound that can be used in the treatment of the pathogen(s) described herein. In some embodiments the treatment compound is an anti-infective, e.g., an antiviral or antibiotic compound. In some embodiments, the treatment compound described herein is selected from the group consisting of fluoroquinolones (e.g., ciprofloxacin), tetracyclines (e.g., doxycycline), macrolides (e.g., azithromycin) and cephalosporins (e.g., ceftriaxone). In some embodiments, the treatment compound described herein is used in adjuvant therapy of an infection such as a pain medication, immunomodulatory compound, anti-inflammatory compound, lubricant altering compound or anticonvulsant compound.

“a,” “an,” and “the” are used herein to refer to one or to more than one (i.e., to at least one, or to one or more) of the grammatical object of the article.

“or” should be understood to mean either one, both, or any combination thereof of the alternatives.

“and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “about” or “approximately”, as used herein, refer to “within 20%”, more preferably “within 10%”, and even more preferably “within 5%”, of a given value or range.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of”.

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

The invention further relates to the following items:

1. A method for detecting the presence of at least two pathogens in a sample, the method comprising the steps of:
a) separating the sample into at least two subsamples;
b) amplifying at least three different target nucleic acid sequences in the subsamples, wherein
   i) at least one target nucleic acid sequence is amplified in each subsample; and
   ii) at least two of the target nucleic acid sequences are identification nucleic acid sequences, wherein each of the identification nucleic acid sequences is characteristic for a pathogen; and
      1.) at least one of the target nucleic acid sequences is a variant target sequence, wherein the variant target sequence is indicative of at least one mutation of at least one of the pathogens; and/or
      2.) at least one of the target nucleic acid sequences is an identity specifying target sequence, wherein at least one of the identification nucleic acid sequences is characteristic for a plurality of subgroups of a pathogen and the identity specifying target sequence is comprised in a subgroup of at least one of the pathogens;
c) determining the presence of amplification products of the target nucleic acid sequences in the subsamples; and
d) detecting at least two pathogens in a sample based on the amplification products determined in c), wherein presence of an amplification product is an indication of a presence, an absence and/or a quantity of one or more pathogens in the sample.

2. The method of item 1, wherein separating the sample according to step a) comprises the substeps of:
1.) accepting a cartridge having a sample port assembly containing the sample;
2.) enriching the nucleic acid to generate an enriched nucleic acid solution; and
3.) separating the sample into at least two subsamples by distributing the enriched nucleic acid solution to two or more assay chambers within the cartridge.

3. The method of item 2, wherein amplifying at least three different target nucleic acid sequences according to step b) comprises performing an amplification reaction within each one of two or more assay chambers in the cartridge while simultaneously detecting amplification product.

4. The method of any one of items 1 to 3, wherein amplifying at least three different target nucleic acid comprises cycling of temperatures, preferably a two-step polymerase chain reaction and a denaturation temperature below 96° C., preferably below 91° C.

5. The method of any one of items 1 to 4, wherein amplifying at least three different target nucleic acid sequences comprises combining each subsample with at least one dried amplification reagent and at least one dried probe for detection, preferably with at least one dried primer, at least one dried probe and a plurality of dried nucleotides.

6. The method of any one of items 1 to 5, wherein determining the presence of amplification products of the variant target sequence comprises comparing the presence of the amplification product of the variant target sequence to the presence of the amplification product of the identification nucleic acid sequence of the same pathogen.

7. The method of any one of items 1 to 6, wherein the variant target sequence comprises an rRNA sequence or an rRNA encoding sequence.

8. The method of any one of items 1 to 7, wherein separating the sample into at least two subsamples comprises separating the sample into at least 3, at least 4, or at least 5 subsamples.

9. The method of any one of items 1 to 8, wherein at least 1, at least 2, at least 3, at least 4, at least 5, at least 6, at least 7, at least 8, at least 9 or at least 10 additional target nucleic acid sequence(s) is/are determined.

10. The method of item 9, wherein the additional target nucleic acid sequence(s) comprise(s) at least one variant control nucleic acid sequence that is amplified in b).

11. The method of item 9 or 10, wherein the additional target nucleic acid sequence(s) comprise(s) at least one confirmation identification nucleic acid sequence that is amplified in b), wherein each confirmation identification nucleic acid sequence is a sequence comprised in the same pathogen as at least one identification nucleic acid sequence.

12. The method of any one of items 9 to 11, wherein the additional target nucleic acid sequence(s) comprise(s) at least 1, or at least 2 further variant target sequence(s).

13. The method of any one of items 1 to 12, wherein the sample comprises a urine sample, a vaginal swab, a urethral swab, a cervical swab, an anal swab, a rectal swab and/or a pharyngeal swab.

14. The method of any one of items 1 to 13, wherein the pathogens are STIs pathogens.

15. The method of any one of items 1 to 14, wherein the pathogens are bacterial pathogens.

16. The method of item 15, wherein the variant target sequence(s) comprise(s) an antibiotic susceptibility variant target sequence.

17. The method of any one of items 14 to 16, wherein the pathogens comprise or consist of Chlamydia trachomatis and Neisseria gonorrhoeae.

18. The method of item 17, wherein at least one subgroup of the pathogen comprises LGV-causing Chlamydia trachomatis.

19. The method of any one of items 1 to 18, wherein a) the amplification reagents comprise at least; and/or b) determining the presence of amplification products, involves binding of at least:
1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 sequences selected from the group of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.

20. The method of item 19, wherein amplifying at least three different target nucleic acid sequences involves binding of at least 1, 2, 3, 4, 5, 6, 7, 8, 9, 10, 11, 12, 13, 14, 15, 16, 17 or 18 sequences selected from the group of: SEQ ID NO: 1, SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 14, SEQ ID NO: 15, SEQ ID NO: 18, SEQ ID NO: 19, SEQ ID NO: 22, SEQ ID NO: 23, SEQ ID NO: 25, SEQ ID NO: 26, SEQ ID NO: 28 and SEQ ID NO: 29.

21. The method of any one of items 18 to 20, wherein
a) at least one identification nucleic acid sequence and at least one identity specifying target sequence are amplified in the same subsample; and/or
b) at least two variant target sequences are amplified in the same subsample.

22. The method of item 21, wherein
a) at least two identification nucleic acid sequences and at least one identity specifying target sequence are amplified in the same subsample; and/or
b) at least one identification nucleic acid sequence and at least two identity specifying target sequences are amplified in the same subsample.

23. The method of any one of items 20 to 22, wherein amplifying at least three different target nucleic acid sequences involves the presence of at least two or at least three primer pairs
a) selected from the group consisting of:
   i) SEQ ID NO: 1 and SEQ ID NO: 2;
   ii) SEQ ID NO: 25 and SEQ ID NO: 26; and
   iii) SEQ ID NO: 28 and SEQ ID NO: 29;
wherein the at least two or at least three primer pairs are present in the same subsample; and/or
b) selected from the group consisting of:
   i) SEQ ID NO: 4 and SEQ ID NO: 5
   ii) SEQ ID NO: 22 and SEQ ID NO: 23
   iii) SEQ ID NO: 7 and SEQ ID NO: 8,
wherein the at least two or at least three primer pairs are present in the same subsample.

24. The method of any one of items 19 to 23, wherein determining the presence of amplification products, involves presence of
a) SEQ ID NO: 12 and SEQ ID NO: 13 in the same subsample;
b) SEQ ID NO: 16 and SEQ ID NO: 17 in the same subsample; and/or
c) SEQ ID NO: 20 and SEQ ID NO: 21 or SEQ ID NO: 34 and SEQ ID NO: 21 in the same subsample.

25. The method of any one of items 1 to 24, wherein determining the presence of amplification products comprises an optical measurement, preferably an optical measurement of a fluorescent labeled probe.

26. The method of item 25, wherein the optical measurement comprises color compensation filtering.

27. A cartridge comprising sample port assembly, two or more assay chambers and the reagents for performing steps a) to c) of the method of items 2 to 26.

28. A kit comprising the cartridge of item 27 and a cartridge reader for detecting the presence of at least two pathogens in a sample according to the method of items 2 to 26.

While embodiments of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

### BRIEF DESCRIPTION OF FIGURES

FIG. 1: Schematic cartridge workflow.

FIG. 2: Fluorophore Spectra of 6-FAM, 6-HEX and ATTO Rho101 allowing multiplexed qPCR.

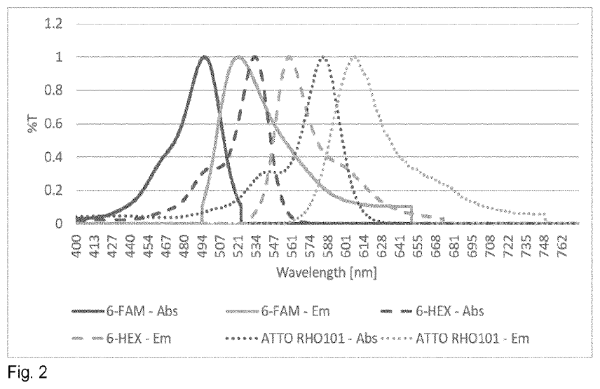

FIG. 3A) Mean Ct-values of NG species detection oligos (SEQ ID 1-3 & SEQ ID 4-6, FAM channel) run on 400cp/rct gDNA of WHO NG reference strains, measured in duplicates. Variation of Ct-values despite equal amount of gDNA input stems from quantification variations as trends across the different WHO strains are the same for both NG detection oligomixes. B) Mean Ct-values of CT species detection oligos (SEQ ID 7-9) run on 400cp/rct gDNA of all CT serovars (A-L), measured in duplicates.

FIG. 4: Mean Ct-values of LGV and nonLGV detection oligos (SEQ IDs 22-30) on 400cp/rct gDNA LGV-1, 4000cp/rct gDNA LGV-3 and 4000cp/rct gDNA nonLGV serovar E, FAM and SUN channels. All three oligomixes are specific for their target: The LGV specific oligos amplified on both LGV templates but did not produce a signal on nonLGV template. The nonLGV specific oligos amplified well on nonLGV template but did not produce a signal of any of the two LGV templates.

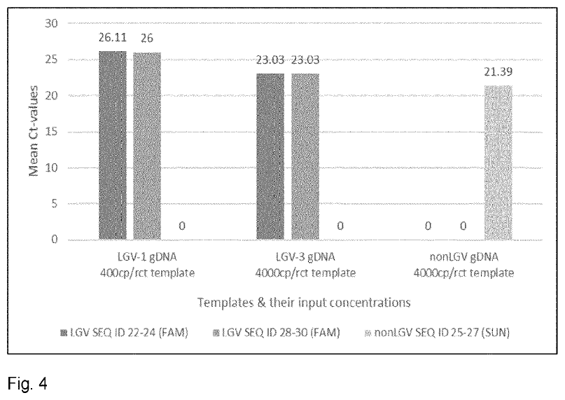

FIG. 5: Amplification signals of NG species detection oligos (SEQ ID 1-3) and CT species detection oligos (SEQ ID 7-9) on 4000cp/rct gDNA of CT serovar E and 4000cp/rct gDNA of NG WHO-F WT, SUN and ATTO-Rho101 channels combined.

(i) Positive control for NG (SUN), (ii) Positive control for CT (ATTO-Rho101), (iii) No NG detection (SUN) on CT nonLGV template, (iv) No CT detection (ATTO-Rho101) on NG template. While both oligomixes worked well on their respective templates (i.e., positive controls), no cross-reactivity between the two species detection oligomixes was observed, showing that the two species detection oligomixes are target specific: the CT detection oligos do not amplify on NG template and the NG detection oligos do not amplify on CT template.

FIG. 6: AMR 23s rRNA C2611T detection primer/probes (SEQ ID 14-17) run on 4000cp/rct E. coli gDNA (i.e., off-target) and on 4000cp/rct gDNA of target NG template in duplicates. (i) Amplification on target template NG 23s rRNA C2611T (MUT), (ii) No amplification on E. coli gDNA template. The figure shows the amplification signals in the SUN channel (MUT signal, SEQ ID 17). No amplification on E. coli was observed with this primer/probe combination.

FIG. 7: A) NG detection oligos (SEQ ID 1-3 and 4-6) on the target template (NG gDNA) and on the Neisseria meningitidis (NM) off-target template (NM gDNA). (i) NG detection oligos on NG target template, (ii) NG detection oligos on NM off-target template. Both oligo combinations do not amplify on the NM off-target template (i.e., no signal and no Ct-value obtained). B) General CT detection oligos (SEQ ID 7-9, ATTO-Rho101 channel) on 4000cp/rct of gDNA of one of the most-closely related off-targets Chlamydia pneumoniae in comparison to a positive control consisting of a published primer/probe set (Ehricht, R.; Slickers, P.; Goellner, S.; Hotzel, H. & Sachse, K. Optimized DNA microarray assay allows detection and genotyping of single PCR-amplifiable target copies, Molecular and Cellular Probes, Elsevier BV, 2006, 20, 60-63) detecting Chlamydia spp. on the same template. (i) Positive control: amplification on C. pneumonia gDNA, (ii) No amplification on C. pneumonia gDNA template with CT oligos SEQ ID No 7-9.

FIG. 8: AMR 23s rRNA A2059G detection primer/probes (SEQ ID 10-13) run on 4000cp/rct of the WT specific target gDNA and the MUT specific target gDNA respectively. (i) Positive controls for both, WT and MUT detection (FAM & SUN), (ii) No WT signal (FAM) on MUT template, (iii) No MUT signal (SUN) on WT template. Target specific signals were obtained for both WT (SEQ ID 12, FAM channel) and MUT (SEQ ID 3, SUN channel) probes (i.e., positive controls). No MUT signal was detected on the WT template and vice versa.

FIG. 9: Exemplary cartridge with 3 reagent containers (i) and 5 detection wells (ii). The sample is inserted with a fixed volume pipette into the sample port (iii) and DNA is extracted automatically. The eluate reconstitutes a lyophilized bead in the homogenization chamber (iv) containing PCR reagents and is distributed into the detection wells.

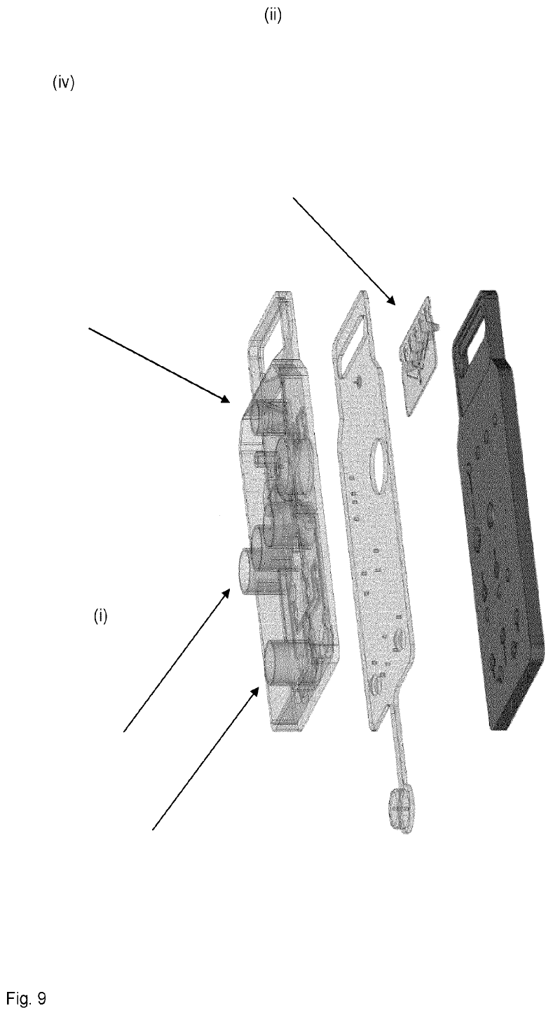

### EXAMPLES

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention.

### Example 1: CT and NG Assay with Discrimination of LGV and Three AMR Related SNPs

### Assay

The assay for Chlamydia trachomatis and Neisseria gonorrhoeae (CT/NG) includes besides species detection also the CT subspecies lymphogranuloma venereum (LGV) and three markers on the NG genome linked to antibiotic resistances (Azithromycin, Ciprofloxacin) (Table 1).

CT species detection is ensured by dual target, including one primer/probe combination that detects CT of all sub-species (serovars A-L3), two primer/probe combinations which only detect the sub-species CT LGV (serovars L1-L3), and one primer/probe combination which only detects the sub-species CT nonLGV (serovars A-K).

NG species detection uses also two target regions with two different NG specific primer/probe combinations targeting two different target regions in the NG genome.

A potential phenotypic susceptibility to Azithromycin is determined by testing for two single nucleotide polymorphisms on the 23s rRNA associated with azithromycin (AZM) resistance (C2611T, associated with low-level AZM resistance; A2059G, associated with high-level AZM resistance).

A decreased susceptibility to Ciprofloxacin (CIP) is determined through detection of the fluoroquinolone resistance—associated gyrA-S91F mutation.

For any SNP genotyping test probes detecting the reference genome sequence (WT) and the variant (MUT) are used in parallel eliciting two distinct fluorescent signals.

In total the assay of this example consists of primer/probes detecting 13 genetic target regions including an internal control, where the internal control can be any nucleic acid target sequence that does not amplify on the other primer and probes in the assay. These primer/probe sets are distributed into five detection wells. This assay uses 3 different fluorophores in each well assay. The assay layout is shown in Table 2.

### Primer and Probe Design

For the design of species and sub-species-specific detection oligos the following steps were performed:

1. 237 publicly available and cleaned CT and 775 NG genome assemblies were retrieved from (publicly) available databases (NCBI and JGI “Genome Portal” for inclusivity database; NCBI only for the exclusivity database) and used as a basis for the bioinformatic analyses.

2. Genomic regions which are present in all genomes of the target species (inclusivity database) were found using open-source software and scripts.

3. The results from the inclusivity database were compared to an exclusivity database (i.e., genome assemblies of 149 off-targets) and to the Blast databases. Regions with high sequence similarity between the different databases were discarded.

4. Quality checks were conducted and detailed information on remaining, target specific genome regions (i.e., repetitive regions, annotation information, etc) was compiled/computed.

5. These bioinformatic analyses resulted in 24 NG specific regions (i.e. sequences of 105-580 bp length with up to 24% mismatch to the off-target sequences) and in 75 CT specific regions (i.e. sequences of 102-650 bp length with up to 100% mismatch to off-target sequences). The most specific regions were selected for primer and probe design for which publicly available tools were used. Each primer/probe combination was then double-checked concerning off-target hits, amplicon size, etc. through (manual) sequence alignments and using BLAST.

6. For NG, 8 different primer/probe combinations were selected, and for CT, 9 different primer/probe combinations were selected and wet-lab tested (Ct-value and signal intensity at target temperature, PCR efficiency, target specificity, multiplexing capacity, robustness), which eventually led to the selection of the oligoset described in Table 3.

7. For the design of the AMR detection oligos the target genome regions were defined by the known genomic resistance markers (SNPs). This made it impossible to entirely avoid amplification/matches with some off-targets, especially for the markers in the 23s rRNA.

The primer and probes determined are SEQ ID 1-30 listed in Table 3 A and B.

The detection method used in the assay of this example is quantitative PCR using hydrolysis probes. For species detection and LGV/nonLGV discrimination specific primers were designed that only amplify a target region on the correct genomic DNA fragment. Fluorescently labeled probes serve as an additional layer for specificity and elicit the signal. For the 3 Antimicrobial resistance markers the primers are capable of amplifying the target region in both WT and MUT strains. Specificity for either WT or MUT is only facilitated by a small difference in the probe sequence together with molecular features that alter base pair stability between probe and target sequence.

### Results

Data presented here has been obtained through qPCR reactions run on a qTOWER, Analytic Jena with a standard two-step qPCR protocol with 45cycles, denaturation temperatures of 95-98° C. and annealing temperatures ranging from 54-63° C.

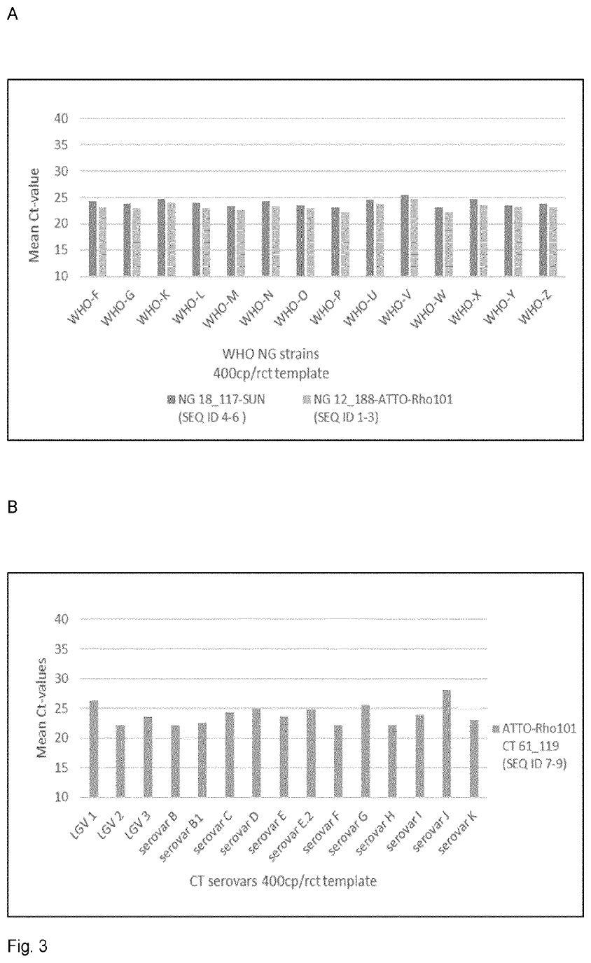

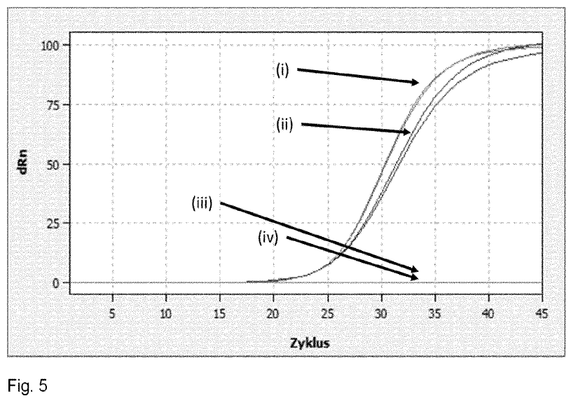

The species and sub-species detection primer/probes (i.e. oligomixes) were tested on gDNA of all CT serovars (A-L) and WHO NG strains, respectively. All CT serovars and NG WHO strains were detected by the respective species/sub-species detection oligos. No cross-reactivity between target species has been observed (FIG. 3 and FIG. 5).

Oligomixes of well 1 and well 5 (Table 2) detect the species CT and NG as well as the sub-species CT LGV/nonLGV. The oligomixes were run on the respective single templates and on a template mix containing gDNA of all 3 target species/sub-species of equal amount. The Ct-value ranges across 10 identical qPCR runs were in an expected range of <1Ct (with the exception of the LGV detection oligos SEQ ID 28-30 where the delta-Ct was slightly increased) for both, single template input and mixed template input. The delta-Ct values between single and mixed template input were also ≤1Ct, with the exception of SEQ ID 7-9 (general CT detection) where the Ct-values were decreased in the template mix compared to the single template. The latter is because this oligomix detects LGV as well as nonLGV template, therefore having more template available in the template mix than in the single template wells, resulting in a decreased Ct-value (Table 4).

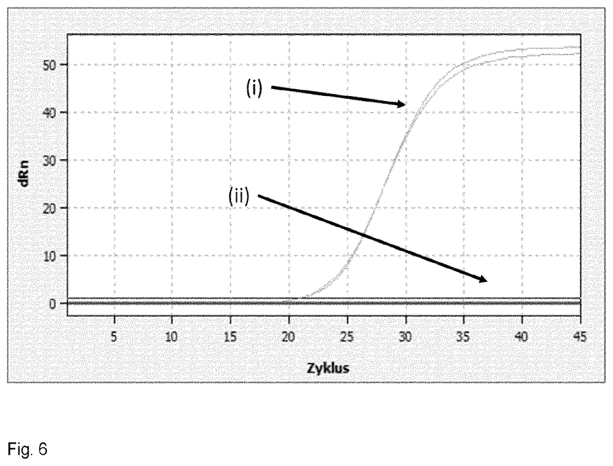

The AMR detection oligos (wells 2-4, Table 2) were tested on gDNA of the corresponding NG WHO strains (well characterized NG reference strains carrying or not carrying the respective genomic resistance markers). When the two probes of each target—i.e., WT and MUT probes—are combined in an oligomix, the amplification is target SNP specific (example shown in FIG. 6). However, occasionally there can be a raw signal on the MUT template that requires comparison of Ct and intensity between WT and MUT probe by the detection algorithm of the optical instrument to distinguish from a true positive signal. As a risk mitigation strategy, the following considerations were made: The MUT probe is specific, meaning the mutation conferring AMR resistance will neither be missed by the assay, nor will it be present if there is no resistance. The additional presence of the WT signal on top of the MUT signal in case of a resistant strain does not disturb as it is not relevant for treatment decision and therefore, in case the optical algorithm fails, no harm is done to the patient.

Off-targets in vitro tested: Neisseria polysacchareae, Neisseria cinerea, Neisseria lactamica, Neisseria oralis, Neisseria perflava, Neisseria flavescence, Neisseria meningitidis, Chlamydia suis, Chlamydia pneumoniae, Human gDNA, E. coli, Mycoplasma genitalium.

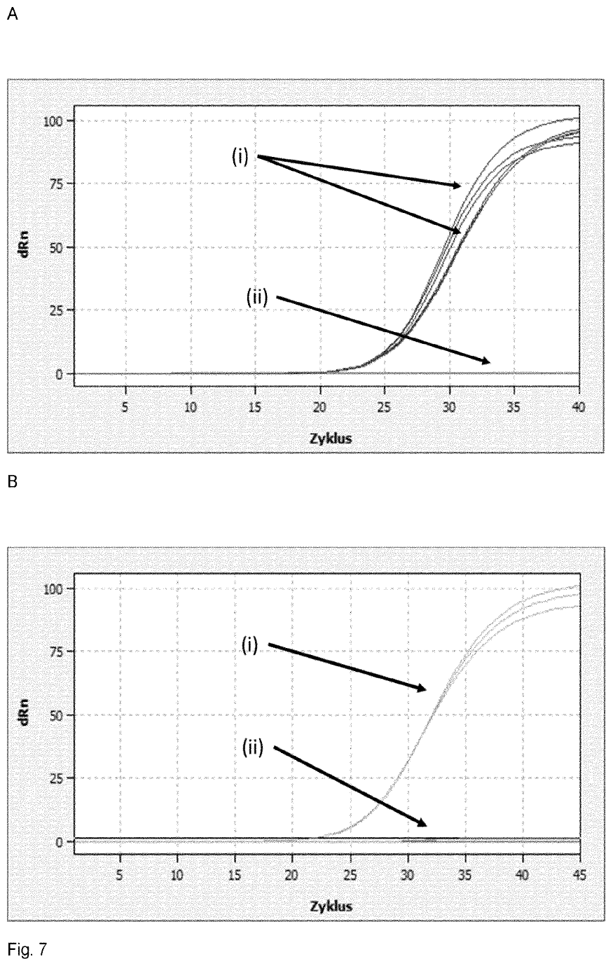

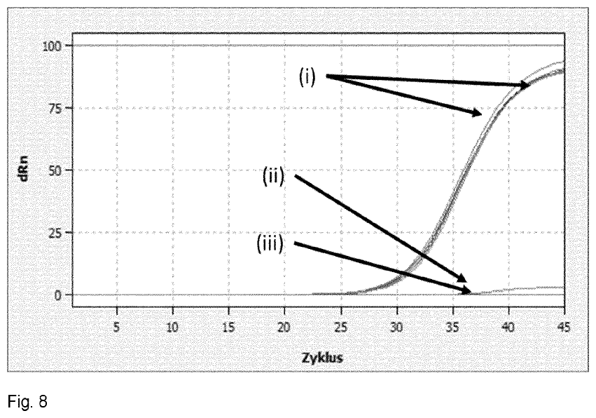

All oligos were tested singleplex (and partially multiplexed) on 1E6 & 1E5cp/ml input gDNA of the above listed off-targets (E. coli also 1E8cp/ml). A positive control was included (i.e., target gDNA). Examples are shown in FIG. 7 and FIG. 8.

While the species and sub-species detection oligos are NG/CT specific, the WT-AMR detection oligos can give signals, especially the oligos detecting resistance markers in the 23s rRNA, due to the conserved sequence across species. The AMR results are therefore coupled to the species detection results and only if NG is detected in a sample, the AMR results will be evaluated. For some off-targets the signal intensities and Ct-values differ significantly between target and off-target template, therefore, for these cases the analysis algorithm will be adapted to exclude “false positive” detections.

### Example 2: Integration into a Cartridge

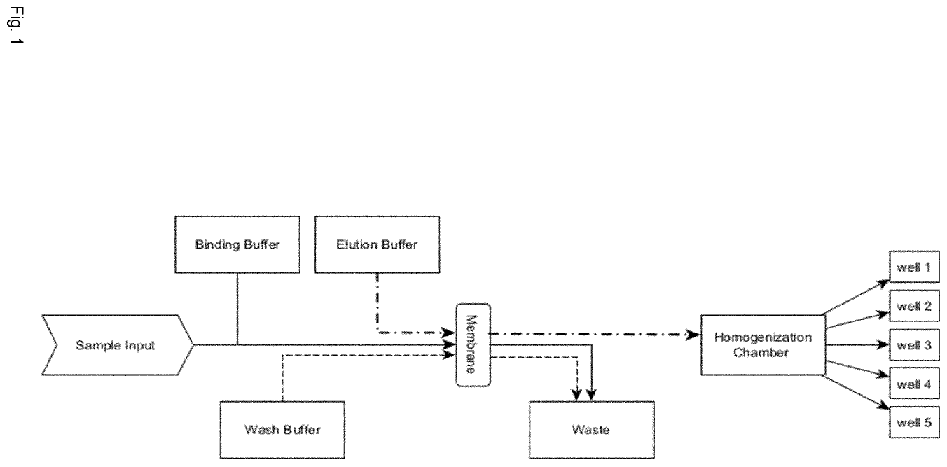

Integration of the assay described in example 1 into a fully contained cartridge requires reagent storage, fluid control and automated DNA extraction. The following method describes an exemplary workflow as shown in FIG. 1.

Input is a defined amount of liquid transport medium that contains a specimen of a urine, vaginal, cervical, urethral, rectal and/or pharyngeal swab. The sample input volume in this example is 400 ul and can be facilitated by using a fixed volume transfer pipette.

The sample is mixed in the cartridge with an equal amount or more of binding buffer that contains any chaotropic agent (for example but not limited to Guanidinium thiocyanate, Guanidinium hydrochloride). The chaotropic agent lyses the bacteria, releases the target DNA and provides the conditions for DNA extraction.

The sample/binding buffer mix is pushed through a silica membrane (glass fiber) where DNA is captured under the chaotropic effect of the binding buffer. Nucleic acids bind to silica in the presence of a chaotropic agent. This binding is established through the disruption of the hydration shell of the nucleic acid by the chaotropic anion in an aqueous solution, which weakens the hydrogen bonding network and elicits hydrophobic interactions with the silica membrane. The capture column in the cartridge can be any silica membrane (e.g., GF/F, Whatman™; GC-50, Advantec). In this example a single layer glass fiber membrane with nominal poresize of 1.6 μm was used (APFA, Merck Millipore).

The following wash step removes residuals of sample or binding buffer that could inhibit the later PCR reaction.

Elution buffer is pushed through the filter and releases the target DNA from the silica membrane. DNA is released from the silica under low salt concentration reversing the binding effect, higher pH supports the reversing effect. For elution a specific buffered salt solution is used which optimally supports qPCR detection.

Eluate is collected in a homogenization chamber with defined volume where it reconstitutes a lyophilized bead containing reagents for the qPCR detection and is then distributed into five distinct detection wells. Each well contains a target specific set of detection primer and probes that were dried into the well and are reconstituted with the eluate (see Table 2).

The instrument then performs a qPCR in the wells by cycling through a heating/cooling profile and optical measurement of a detectable fluorescent signal released by the probe.

### Internal Process Control

The assay may contain an internal process control (IC). The IC can be any unrelated target sequence that does not interfere or cross react with the primer and probes of the assay or with the target pathogens.

The IC is supplied in a constant concentration in the binding buffer and passes through the entire DNA extraction and amplification process (binding to the capture membrane and elution thereof, passing through the distribution chamber, serving as a template for PCR and eliciting a fluorescent signal that is read by the instrument). The IC controls for failures that can occur in any step of the process target DNA passes through. If the IC is detected in the end all the fluidic and biochemical steps inside the cartridge occurred as intended.

### Fluid Control

The sample and reagents are controlled in the cartridge through a system of active and passive valves. Driving force in this example is generated by a syringe pump that is manipulated by external actuators. Other embodiments include for example diaphragm pumps that are integrated into the cartridge.

### Amplification

The extracted target DNA is amplified in a two-step polymerase chain reaction (PCR) and fluorescence is measured during amplification. The standard PCR parameters used are a denaturation temperature of 90° C. and an annealing/elongation temperature of 60° C. over 45 cycles. However, depending on the primer/probe selection and their specific melt temperatures, parameters can be adapted. The dwell times can be chosen from 20 s denaturation and 10 s annealing/elongation down to a dwell time of 1 s. Depending on heating/cooling rates and the instrument's scan time, the total time for 45 cycles can be <30 min. Total time might even be reduced to 20 min by for example lowering the denaturation temperature by 5° C., which reduces cooling/heating time, by improved heating and cooling ramp rates, by measuring only a subset of cycles and interpolating the datapoints or by reducing total cycle numbers.

### Optical Detection

Presence of a pathogen is detected by optical measurement of the fluorescent labeled probes that are specific for each target. The probes are excited with their individual absorption wavelength and the emitted light triggers a photomultiplier or a Multi-Pixel Photon Counter (MPPC).

The detection unit cycles through all five wells and measures sequentially each channel. Irradiation and detection are performed simultaneously ensured by different optical excitation and emission filters for the incoming and outgoing light minimizing accidental signal crosstalk.

The detection unit detects the light intensity proportional to the number of probes bound to amplification products during PCR. The cycle where the signal exceeds background level significantly is calculated by the detection algorithm. Together with the final signal intensity, these two parameters are evaluated for the result interpretation.

Since the assay features multiplexed reactions, crosstalk may occur due to overlapping of the fluorophore's spectra. Appropriate filter selection for optical illumination and detection path reduces the effect to a minimum. Additionally, a color compensation can be applied by the detection algorithm, which anticipates signal of each fluorophore excited and detected by the opposite channels.

The example assay uses three fluorophore groups in combination with adequate fluorescent quenchers and excitation and emission filters (Table 2).

### Detection Algorithm

Light intensity is measured during every cycle for each well and fluorophore (target). After baseline normalization, a threshold cycle (Ct value) is calculated for each curve and the final intensity relative to the baseline is determined. The Ct value is the cycle, where the signal significantly exceeds the background signal.

Based on these two parameters, Ct value and intensity, the algorithm determines if the signal is considered positive or not. Since there can be signal artefacts or unspecific amplification with increasing cycle number in any qPCR reaction, there can be implemented a Ct-threshold and an intensity-threshold that needs to be met. If either the Ct value is above the Ct-threshold, or the final intensity is below the intensity-threshold the signal is considered negative.

### Cartridge Run and Results

To demonstrate the integration into a cartridge, negative clinical sample from cervical swabs (Abbott Multi-collect specimen collection kit) was spiked with either 1E5cp/ml NG gDNA (WHO F) and nonLGV CT DNA (Serovar E) or with 1E5cp/ml NG DNA and LGV CT DNA (LGV-3). Wash and elution buffer was Tris-Hydrochlorid (Tris HCl) in this example. Two technical replicates were produced for each combination and the sample was processed in a demonstrating cartridge. All process steps (liquid control, binding, washing and elution) were executed automatically. A syringe drive and a cartridge drive provided the pressure for liquid control and valve actuation within the cartridge. Primer and probe combinations of well 1 or well 5 (see Table 2) were added to the eluate and mixed with mastermix (5star™, biotechrabbit). Using in total 6 target regions allow to detect NG and CT with dual target while simultaneously discriminating the CT subspecies LGV and nonLGV.

Detection was performed on a commercially available qPCR machine (qTower³ touch, Analytic Jena). The mastermix and/or the primer and probes were added manually for the detection but can also be provided in a lyophilized or dried state in the cartridge.

NG and CT species detection oligos amplified in all samples as dual target. Furthermore, the LGV specific oligos amplified on LGV templates but did not produce a signal on nonLGV template. The nonLGV specific oligos amplified well on nonLGV template but did not produce a signal of the LGV template (see Table 5).

### Example 3

Mutations resulting in antibiotic resistances are known in the art. Table 6 demonstrates relevant mutations and the corresponding drug resistance.

### Example 4

Comparison to known methods are demonstrated in Table 7. The means and methods provided herein enable a surprisingly efficient and precise detection.

TABLE 1
Overview of gene regions used for detections in CT and NG

| | Species/ | | |
|---|---|---|---|
| | Subspecies/ | | Mutation/ |
| Target | AMR | Gene region | Wildtype |
| CT | Species | locus_tag CT_783 | n/a |
| | Subspecies | Gene = pmpC | n/a |
| | nonLGV | locus_tag = CT_414 | |
| | Subspecies LGV | locus_tag = CTL2C_222 | n/a |
| | Subspecies LGV | gene = mutL; | n/a |
| | | locus_tag = CTL2C_437 | |
| NG | Species | locus_tag = NGO0125a | n/a |
| | Species | locus_tag = NGO0124/5 | n/a |
| | Resistance AZM | 23s rRNA (rrl) | C2611T/ |
| | | | C2611C |
| | Resistance AZM | 23s rRNA (rrl) | A2059G/ |
| | | | A2059A |
| | Resistance CIP | gyrA | S91F/S91S |

TABLE 2
Assay Layout

| | Target1-FAM | Target2-HEX/SUN | Target3 -ATTO-Rho101 |
|---|---|---|---|
| Well 1 | Sub-species detection | Species detection | Species detection |
| | Chlamydia trachomatis - LGV | Neisseria gonorrhoeae | Chlamydia trachomatis |
| | SEQ ID 22-24) | (SEQ ID 4-6) | (SEQ ID 7-9) |
| Well 2 | Antimicrobial resistance - Azithro | Antimicrobial resistance - Azithro | empty |
| | C2611C WT (SEQ ID 14-15, 16) | C2611T MUT (SEQ ID 14-15, 17) | |
| Well 3 | Antimicrobial resistance - Cipro gyrA | Antimicrobial resistance - Cipro gyrA | empty |
| | S91F - MUT (SEQ ID 18-19) | S91S - WT (SEQ ID 18-19, 20) | |
| Well 4 | Antimicrobial resistance - Azithro | Antimicrobial resistance - Azithro | Internal control |
| | A2059A WT (SEQ ID 10-12) | A2059G - MUT (SEQ ID 10-11, 13) | (SEQ ID 31-33) |
| Well 5 | Sub-species detection | Sub-species detection | Species detection |
| | Chlamydia trachomatis - LGV2 | Chlamydia trachomatis - nonLGV | Neisseria gonorrhoeae |
| | SEQ ID 28-30 | (SEQ ID 25-27) | (SEQ ID 1-3) |

TABLE 3
Oligo information: A

| | | | | | | Target | Amplicon |
|---|---|---|---|---|---|---|---|
| Primer | Fluorphore | Probetype | Quencher | Sequence ID | Sequence (5′ > 3′) | (gene, region...) | (bp) |
| NG_12_188_F | n/a | n/a | n/a | SEQ ID NO: 1 | CGCGCAAGTTTACGGGAAT | locus_tag = NGO0125a; | 84 |
| NG_12_188_R | n/a | n/a | n/a | SEQ ID NO: 2 | GGATGCGGATTTCCTGCTTT | product = peptidase | |
| NG_12_188_P | ATTO- | TaqMan | 3IAbRQSp | SEQ ID NO: 3 | CAAACACGCGTTTCGAGGCAGG | | |
| | Rho101 | | | | | | |
| NG_18_117_F | n/a | n/a | n/a | SEQ ID NO: 4 | AACTGCCAATGCGGAGAA | locus_tags = | 96 |
| NG_18_117_R | n/a | n/a | n/a | SEQ ID NO: 5 | GCTTTCCAGTGTTTCAGGTAATG | NGO0124/NGO0125 | |
| NG_18_117_P | SUN | TaqMan | ZEN ™ (internal) | SEQ ID NO: 6 | CGTGCAACTGTTGATCGGGAGTTT | product = hypothetical | |
| | | | 13IABkFQ | | | proteins | |
| | | | (= IB®FQ) | | | | |
| CT_61_119_F | n/a | n/a | n/a | SEQ ID NO: 7 | AAATCCGTGATGAAGTGCTAAG | locus_tag = CT_783 | 96 |
| CT_61_119_R | n/a | n/a | n/a | SEQ ID NO: 8 | ACTCTGTATGACGAGGGAAATC | | |
| CT_61_119_P | ATTO- | TaqMan | 3IAbRQSp | SEQ ID NO: 9 | TGTTGCTGACCAATTTGTGTGCGT | | |
| | Rho101 | | | | | | |
| 23s_A2059_F | n/a | n/a | n/a | SEQ ID NO: 10 | GAAGTTGAAGTGGTTGTGAAGA | NG 23s rRNA | 84 |
| 23s_A2059_R | n/a | n/a | n/a | SEQ ID NO: 11 | TCCAATGCAAAGCTACAGTAAA | | |
| 23s_A2059_P_WT | FAM | Affinity Plus | 13IABkFQ | SEQ ID NO: 12 | AGA+CGG+A+A+AG+A+CC | | |
| | | | (= IB®FQ) | | | | |
| 23s_A2059_P_MUT | SUN | Affinity Plus | IB®FQ) | SEQ ID NO: 13 | AC+GG+A+G+AG+ACC | | |
| 23s_C2611_F | n/a | n/a | n/a | SEQ ID NO: 14 | CAAGGGTATGGCTGTTCG | NG 23s rRNA | 117 |
| 23s_C2611_R | n/a | n/a | n/a | SEQ ID NO: 15 | CCCGTCAAACTTCCAACG | | |
| 23s_C2611_P_WT | FAM | Affinity Plus | IB®FQ) | SEQ ID NO: 16 | TA+G+G+GAC+C+AA | | |
| 23s_C2611_P_MUT | SUN | Affinity Plus | 13IABkFQ | SEQ ID NO: 17 | AGATA+G+A+GA+C+CAA | | |
| | | | (= IB®FQ) | | | | |

TABLE 3B

| | Fluor- | Probe- | | | | Target | Amplicon |
|---|---|---|---|---|---|---|---|
| Primer | phore | type | Quencher | Sequence ID | Sequence (5′-3′) | (gene, region; | (bp) |
| gyrA_S91_F | n/a | n/a | n/a | SEQ ID NO: 18 | CGTCATCGGTAAATACCACC | NG gyrA | 75 or 76 |
| gyrA_S91_R | n/a | n/a | n/a | SEQ ID NO: 19 | GCGAAATTTTGCGCCATA | | |
| gyrA_S91_P_WT | HEX | MGB | MQ530 | SEQ ID NO: 20 | CGGCGATTCCGCAGT | | |
| gyrA_S91_P_WT | HEX | MGB | MQ530 | SEQ ID NO: 34 | ACGGCGATTCCGCAGTT | | |
| gyrA_S91_P_MUT | FAM | MGB | MQ530 | SEQ ID NO: 21 | CGGCGATTTCGCAGT | | |
| 10_308_L_F | n/a | n/a | n/a | SEQ ID NO: 22 | GGGTAACTTGTACATCAGTTGTAGT | locus_tag = CTL2C_222; | 76 |
| 10_308_R | n/a | n/a | n/a | SEQ ID NO: 23 | GCTGGCATCCCTAGTATTAAAGC | product = chlamydia | |
| 10_308_P | FAM | TaqMan | ZENTM (internal) | SEQ ID NO: 24 | ATCCGGATCCACTTCCTGCAGAA | polymorphic membrane protein | |
| | | | 13IABkFQ | | | middle domain protein | |
| | | | (= IB®FQ) | | | | |
| 114_138_F | n/a | n/a | n/a | SEQ ID NO: 25 | GCTATGCTGCGAGTACTGATA | gene = pmpC; | 98 |
| 114_138_R | n/a | n/a | n/a | SEQ ID NO: 26 | AGAAGATGCAGTAACGTCTGAA | locus_tag = CT_414; | |
| 114_138_P | SUN | TaqMan | ZEN ™ (internal) | SEQ ID NO: 27 | AGAAGAGCCTGTCACTTCTTCTTCA | product = Putative outer | |
| | | | 13IABkFQ | | | membrane protein C | |
| | | | (= IB®FQ) | | | | |
| 137_142_F | n/a | n/a | n/a | SEQ ID NO: 28 | CGAGAGACAGCACGAGAGAAA | gene = mutL; | 91 |
| 137_142_R | n/a | n/a | n/a | SEQ ID NO: 29 | GTGCAGCAGCGTGTGTC | locus_tag = CTL2C_437; | |
| 137_142_P | FAM | TaqMan | ZEN™ (internal) | SEQ ID NO: 30 | AGGAGGTAACGGAAAAATTGCAAGGCA | product = DNA mismatch repair | |
| | | | 13IABkFQ | | | protein MutL family protein | |
| | | | (= IB®FQ) | | | | |
| IC_F | n/a | n/a | n/a | SEQ ID NO: 31 | GAATCAGCCCGACTCGTT | 250 bp gBlock with part of | 81 |
| IC_R | n/a | n/a | n/a | SEQ ID NO: 32 | CTGAGCAAGTCCGGAGATAAG | ″plant combinatorial and | |
| IC_P | ATTO- | TaqMan | 3IAbRQSp | SEQ ID NO: 33 | CTCCGACTCACTCCTGCACGAAAC | modular protein-PCMP1″ of the | |
| | Rho101 | | | | | species arabidobsis | |
| | | | | | | thaliana AccNo. LR782546 | |

Table 3A: “+” within a sequence indicates that the following nucleotide is a locked nucleic acid nucleotide (e.g., +A=LNA-A)

TABLE 4
CT-value ranges of 10 identical qPCR repeat runs with primer/probes
of well 1 and well 5 on single and on mixed gDNA target templates

| | | | | | Oligomix - on 3 | | | |
|---|---|---|---|---|---|---|---|---|
| | | Singleplex on correct | | | templates: | | | |
| | | template | | | NG/LGV/CT | | | Delta − Ct |
| Ct-Range across 10 plates | | | | Delta − | | | Delta − | single − |
| Detection | | | | Min − | | | Min − | triple |
| Well | Target and primer/probe | Min | Max | Max | Min | Max | Max | template |
| Well 1 | LGV SEQ ID 22-24 | 21.7 | 22.5 | 0.8 | 20.9 | 21.6 | 0.7 | 0.8 |
| | NG SEQ ID 4-6 | 22.1 | 22.8 | 0.7 | 20.7 | 21.6 | 0.9 | 1.4 |
| | CT SEQ ID 7-9 | 22.6 | 23 | 0.4 | 19.2 | 19.6 | 0.4 | 3.4 |
| Well 5 | LGV SEQ ID 28-30 | 21.5 | 22.6 | 1.1 | 20.8 | 21.6 | 0.8 | 0.7 |
| | nonLGV SEQ ID 25-27 | 22.6 | 23.2 | 0.6 | 22.2 | 22.8 | 0.6 | 0.4 |
| | NG SEQ ID 1-3 | 21.9 | 22.8 | 0.9 | 21.3 | 22.1 | 0.8 | 0.6 |

TABLE 5
Ct values of spiked clinical cervical sample (1E5 cp/ml
NG, LGV or nonLGV gDNA) processed in a demonstration
cartridge with oligomix 1 (A) and 5 (B) (see Table 2).
A

| Pathogen | LGV SEQ ID | NG SEQ ID | (CT SEQ ID 7-9) |
|---|---|---|---|
| gDNA | 22-24 (FAM) | 4-6 (SUN) | (ATTO Rho101) |
| NG + LGV | 29.3 | 25.2 | 28.8 |
| NG + LGV | 29.1 | 25.2 | 28.5 |
| NG + nonLGV | No Ct | 26.1 | 26.5 |
| NG + nonLGV | No Ct | 26.7 | 27 |

B

| Pathogen | LGV SEQ ID | nonLGV SEQ ID | NG SEQ ID 1-3 |
|---|---|---|---|
| gDNA | 28-30 (FAM) | 25-27 (SUN) | (ATTO Rho101) |
| NG + LGV | 29.5 | No Ct | 25.6 |
| NG + LGV | 29.9 | No Ct | 26.0 |
| NG + nonLGV | No Ct | 26.7 | 26.8 |
| NG + nonLGV | No Ct | 27.3 | 27.1 |

TABLE 6
Drug resistance

| | Resistance | | |
|---|---|---|---|
| Gene | marker | Drug resistance | References (both for all 3 markers) |
| gyrA | S91F | Ciprofloxacin | Eyre et al 2017 |
| 23s | A2059G | Azithromycin (high | J Antimicrob Chemother 2017; 72: 1937-1947 |
| rRNA | | level resistance) | doi: 10.1093/jac/dkx067 Advance Access publication |
| 23s | C2611C | Azithromycin (low | 10 Mar. 2017 |
| rRNA | | level resistance) | Unemo & Schaefer 2014 |
| | | | ASM Journals Clinical Microbiology Reviews |
| | | | Vol. 27, No. 3 |
| | | | Antimicrobial Resistance in Neisseria gonorrhoeae in |
| | | | the 21st Century: Past, Evolution, and Future |
| | | | DOI: https://doi.org/10.1128/CMR.00010-14 |

TABLE 7
Comparison to known methods

| | | TTR | AMR | Subspecies | | CT + NG |
|---|---|---|---|---|---|---|
| Platform/Instrument | Company | [min] | NG | CT LGV | POC | Assay |
| Sefunda PoC Instrument | Sefunda AG | 30 | Yes | Yes | Yes | Yes |
| Io System | Binx Health | 30 | No | No | Yes | Yes |
| Visby Medical Test | Visby Medical | 30 | Yes | No | Yes | Yes |
| Vivalytic | Bosch | 30-90 | No | No | Yes | Yes |
| GeneXpert | Cepheid | 90 | No | No | Yes | Yes |
| Real time (e.g. m2000) | Abbott | >60 | No | No | No | Yes |

1. A method for determining whether a sample contains a pathogen, the method comprising the steps of:
a) separating the sample into at least two subsamples;
b) bringing different amplification reagents into contact with at least two of the subsamples for amplifying at least three different target nucleic acid sequences, wherein at least two of the target nucleic acid sequences are identification nucleic acid sequences, wherein each of the identification nucleic acid sequences is characteristic for a pathogen; and
1. at least one of the target nucleic acid sequences is a variant target sequence, wherein the variant target sequence is indicative of at least one mutation of at least one of the pathogens; and/or
2. at least one of the target nucleic acid sequences is an identity specifying target sequence, wherein at least one of the identification nucleic acid sequences is characteristic for a plurality of subgroups of at least one of the pathogens and the identity specifying target sequence is comprised in one of the subgroups of the pathogen;
c) determining the presence or absence of amplification products of the target nucleic acid sequences in the subsamples using at least one detection probe; and
d) determining whether the sample contains a pathogen, wherein presence of an amplification product is an indication of a presence, an absence and/or a quantity of one or more pathogens in the sample.

2. The method of claim 1, wherein separating the sample according to step a) comprises the substeps of:
1. accepting a cartridge having a sample port assembly containing the sample;
2. enriching the nucleic acid to generate an enriched nucleic acid solution; and
3. separating the sample into at least two subsamples by distributing the enriched nucleic acid solution to two or more assay chambers within the cartridge.

3. The method of claim 2, wherein amplifying at least three different target nucleic acid sequences according to step b) comprises performing an amplification reaction within each one of two or more assay chambers in the cartridge while simultaneously detecting amplification product.

4. (canceled)

5. The method of claim 1, wherein bringing amplification reagents into contact with the subsamples for amplifying at least three different target nucleic acid sequences comprises combining each subsample with at least one dried amplification reagent and at least one dried probe for detection, preferably with at least one dried primer, at least one dried probe and a plurality of dried nucleotides.

6. The method of claim 1, wherein determining the presence or absence of amplification products of the variant target sequence comprises comparing the presence or absence of the amplification product of the variant target sequence to the presence or absence of the amplification product of the identification nucleic acid sequence of the same pathogen.

7.-8. (canceled)

9. The method of claim 1, wherein at least 1, at least 2, at least 3, at least 4, at least 5, at least 6, at least 7, at least 8, at least 9 or at least 10 additional target nucleic acid sequence(s) is/are determined.

10. The method of claim 9, wherein
1) the additional target nucleic acid sequence(s) comprise(s) at least one variant control nucleic acid sequence that is amplified in b); and/or
2. the additional target nucleic acid sequence(s) comprise(s) at least one confirmation identification nucleic acid sequence that is amplified in b), wherein each confirmation identification nucleic acid sequence is a sequence comprised in the same pathogen as at least one identification nucleic acid sequence.

11. (canceled)

12. The method of claim 9, wherein the additional target nucleic acid sequence(s) comprise(s) at least 1, or at least 2 further variant target sequence(s).

13. (canceled)

14. The method of claim 1, wherein the pathogens are STIs pathogens, or bacterial pathogens.

15.-16. (canceled)

17. The method of claim 14, wherein the pathogens comprise or consist of Chlamydia trachomatis and Neisseria gonorrhoeae.

18. The method of claim 17, wherein at least one subgroup of the pathogen comprises LGV-causing Chlamydia trachomatis.

19. The method of claim 1, wherein a) the amplification reagents comprise at least; and/or b) determining the presence of amplification products, involves binding of at least:
1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 sequences selected from the group of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.

20. The method of claim 19, wherein the amplification reagents comprise at least 1, 2, 3, 4, 5, 6, 7, 8, 9, 10, 11, 12, 13, 14, 15, 16, 17 or 18 sequences selected from the group of: SEQ ID NO: 1, SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 14, SEQ ID NO: 15, SEQ ID NO: 18, SEQ ID NO: 19, SEQ ID NO: 22, SEQ ID NO: 23, SEQ ID NO: 25, SEQ ID NO: 26, SEQ ID NO: 28 and SEQ ID NO: 29.

21. The method of claim 18, wherein
a) at least one identification nucleic acid sequence and at least one identity specifying target sequence are amplified in the same subsample; and/or
b) at least two variant target sequences are amplified in the same subsample;
or
a) at least two identification nucleic acid sequences and at least one identity specifying target sequence are amplified in the same subsample; and/or
b) at least one identification nucleic acid sequence and at least two identity specifying target sequences are amplified in the same subsample.

22. (canceled)

23. The method of claim 20, wherein the amplification reagents comprise at least two or at least three primer pairs
a) selected from the group consisting of:
i) SEQ ID NO: 1 and SEQ ID NO: 2;
ii) SEQ ID NO: 25 and SEQ ID NO: 26; and
iii) SEQ ID NO: 28 and SEQ ID NO: 29;
wherein the at least two or at least three primer pairs are present in the same subsample;
and/or
b) selected from the group consisting of:
i) SEQ ID NO: 4 and SEQ ID NO: 5
ii) SEQ ID NO: 22 and SEQ ID NO: 23
iii) SEQ ID NO: 7 and SEQ ID NO: 8,
wherein the at least two or at least three primer pairs are present in the same subsample.

24. The method of claim 19, wherein
determining the presence of amplification products, involves presence of
a) SEQ ID NO: 12 and SEQ ID NO: 13 in the same subsample;
b) SEQ ID NO: 16 and SEQ ID NO: 17 in the same subsample; and/or
c) SEQ ID NO: 20 and SEQ ID NO: 21 in the same subsample.

25.-26. (canceled)

27. A cartridge comprising sample port assembly, two or more assay chambers and the reagents for performing steps a) to c) of the method of claim 2.

28. (canceled)

29. A method for diagnosing a subject with an infectious disease induced by a pathogen variant and/or an infection with a pathogen subgroup, the method comprising the steps of:
(I) determining whether a sample of a subject contains a pathogen according to the method of claim 1;
(II) comparing the determination of step (I) with a diagnosis reference value,
(III) determining based on the comparison in step (II) whether the sample is indicative of:
a) i) the presence of a first pathogen and ii) a variant and/or subgroup thereof; and/or
b) i) the presence of a second pathogen and ii) a variant and/or subgroup thereof; and
(IV) diagnosing the subject with an infectious disease induced by at least one pathogen variant and/or an infectious disease induced by at least one pathogen subgroup of at least one pathogen based on the presence in the sample determined in step (III).

30.-31. (canceled)

32. A method of treatment of an infection, the method comprising the step of:
administering to the subject to be treated an effective dose of a pharmaceutical composition, wherein the subject to be treated is
a) a subject from which a sample is determined to contain a pathogen according to the method of claim 1 and wherein the pharmaceutical composition comprises an active therapeutic agent having known activity against the pathogen(s) present in the sample; and/or
b) a subject which is determined to be susceptible to the treatment with an anti-infective agent and wherein the pharmaceutical composition comprises the anti-infective agent.

33. (canceled)

34. A composition comprising reagents for the detection of a pathogen, the composition comprising 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleic acid molecule(s) having the sequence(s) selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.


SEQUENCE LISTING (34 sequences)

SEQ ID NO: 1 - DNA, 19 bp, synthetic construct
  misc_feature 1..19: note = NG_12_188_F
  source 1..19: mol_type = other DNA
cgcgcaagtttacgggaat

SEQ ID NO: 2 - DNA, 20 bp, synthetic construct
  misc_feature 1..20: note = NG_12_188_R
  source 1..20: mol_type = other DNA
ggatgcggatttcctgcttt

SEQ ID NO: 3 - DNA, 22 bp, synthetic construct
  misc_feature 1..22: note = NG_12_188_P
  source 1..22: mol_type = other DNA
caaacacgcgtttcgaggcagg

SEQ ID NO: 4 - DNA, 18 bp, synthetic construct
  misc_feature 1..18: note = NG_18_117_F
  source 1..18: mol_type = other DNA
aactgccaatgcggagaa

SEQ ID NO: 5 - DNA, 23 bp, synthetic construct
  misc_feature 1..23: note = NG_18_117_R
  source 1..23: mol_type = other DNA
gctttccagtgtttcaggtaatg

SEQ ID NO: 6 - DNA, 24 bp, synthetic construct
  misc_feature 1..24: note = NG_18_117_P
  source 1..24: mol_type = other DNA
cgtgcaactgttgatcgggagttt

SEQ ID NO: 7 - DNA, 22 bp, synthetic construct
  misc_feature 1..22: note = CT_61_119_F
  source 1..22: mol_type = other DNA
aaatccgtgatgaagtgctaag

SEQ ID NO: 8 - DNA, 22 bp, synthetic construct
  misc_feature 1..22: note = CT_61_119_R
  source 1..22: mol_type = other DNA
actctgtatgacgagggaaatc

SEQ ID NO: 9 - DNA, 24 bp, synthetic construct
  misc_feature 1..24: note = CT_61_119_P
  source 1..24: mol_type = other DNA
tgttgctgaccaatttgtgtgcgt

SEQ ID NO: 10 - DNA, 22 bp, synthetic construct
  misc_feature 1..22: note = 23s_A2059_F
  source 1..22: mol_type = other DNA
gaagttgaagtggttgtgaaga

SEQ ID NO: 11 - DNA, 22 bp, synthetic construct
  misc_feature 1..22: note = 23s_A2059_R
  source 1..22: mol_type = other DNA
tccaatgcaaagctacagtaaa

SEQ ID NO: 12 - DNA, 13 bp, synthetic construct
  misc_feature 1..13: note = 23s_A2059_P_WT
  modified_base 4: mod_base = OTHER; note = locked nucleic acid C
  modified_base 7: mod_base = OTHER; note = locked nucleic acid A
  modified_base 8: mod_base = OTHER; note = locked nucleic acid A
  modified_base 9: mod_base = OTHER; note = locked nucleic acid A
  modified_base 11: mod_base = OTHER; note = locked nucleic acid A
  modified_base 12: mod_base = OTHER; note = locked nucleic acid C
  source 1..13: mol_type = other DNA
agacggaaagacc

SEQ ID NO: 13 - DNA, 11 bp, synthetic construct
  misc_feature 1..11: note = 23s_A2059_P_MUT
  misc_feature 3: note = /note="locked nucleic acid G"
  misc_feature 5: note = /note="locked nucleic acid A"
  misc_feature 6: note = /note="locked nucleic acid G"
  misc_feature 7: note = /note="locked nucleic acid A"
  misc_feature 9: note = /note="locked nucleic acid A"
  source 1..11: mol_type = other DNA
acggagagacc

SEQ ID NO: 14 - DNA, 18 bp, synthetic construct
  misc_feature 1..18: note = 23s_C2611_F
  source 1..18: mol_type = other DNA
caagggtatggctgttcg

SEQ ID NO: 15 - DNA, 18 bp, synthetic construct
  misc_feature 1..18: note = 23s_C2611_R
  source 1..18: mol_type = other DNA
cccgtcaaacttccaacg

SEQ ID NO: 16 - DNA, 10 bp, synthetic construct
  misc_feature 1..10: note = 23s_C2611_P_WT
  modified_base 3: mod_base = OTHER; note = locked nucleic acid G
  modified_base 4: mod_base = OTHER; note = locked nucleic acid G
  modified_base 5: mod_base = OTHER; note = locked nucleic acid G
  modified_base 8: mod_base = OTHER; note = locked nucleic acid C
  modified_base 9: mod_base = OTHER; note = locked nucleic acid A
  source 1..10: mol_type = other DNA
tagggaccaa

SEQ ID NO: 17 - DNA, 13 bp, synthetic construct
  misc_feature 1..13: note = 23s_C2611_P_MUT
  modified_base 6: mod_base = OTHER; note = locked nucleic acid G
  modified_base 7: mod_base = OTHER; note = locked nucleic acid A
  modified_base 8: mod_base = OTHER; note = locked nucleic acid G
  modified_base 10: mod_base = OTHER; note = locked nucleic acid C
  modified_base 11: mod_base = OTHER; note = locked nucleic acid C
  source 1..13: mol_type = other DNA
agatagagaccaa

SEQ ID NO: 18 - DNA, 20 bp, synthetic construct
  misc_feature 1..20: note = gyrA_S91_F
  source 1..20: mol_type = other DNA
cgtcatcggtaaataccacc

SEQ ID NO: 19 - DNA, 18 bp, synthetic construct
  misc_feature 1..18: note = gyrA_S91_R
  source 1..18: mol_type = other DNA
gcgaaattttgcgccata

SEQ ID NO: 20 - DNA, 15 bp, synthetic construct
  misc_feature 1..15: note = gyrA_S91_P_WT
  source 1..15: mol_type = other DNA
cggcgattccgcagt

SEQ ID NO: 21 - DNA, 15 bp, synthetic construct
  misc_feature 1..15: note = gyrA_S91_P_MUT
  source 1..15: mol_type = other DNA
cggcgatttcgcagt

SEQ ID NO: 22 - DNA, 25 bp, synthetic construct
  misc_feature 1..25: note = 10_308_L_F
  source 1..25: mol_type = other DNA
gggtaacttgtacatcagttgtagt

SEQ ID NO: 23 - DNA, 23 bp, synthetic construct
  misc_feature 1..23: note = 10_308_R
  source 1..23: mol_type = other DNA
gctggcatccctagtattaaagc

SEQ ID NO: 24 - DNA, 23 bp, synthetic construct
  misc_feature 1..23: note = 10_308_P
  source 1..23: mol_type = other DNA
atccggatccacttcctgcagaa

SEQ ID NO: 25 - DNA, 21 bp, synthetic construct
  misc_feature 1..21: note = 114_138_F
  source 1..21: mol_type = other DNA
gctatgctgcgagtactgata

SEQ ID NO: 26 - DNA, 22 bp, synthetic construct
  misc_feature 1..22: note = 114_138_R
  source 1..22: mol_type = other DNA
agaagatgcagtaacgtctgaa

SEQ ID NO: 27 - DNA, 25 bp, synthetic construct
  misc_feature 1..25: note = 114_138_P
  source 1..25: mol_type = other DNA
agaagagcctgtcacttcttcttca

SEQ ID NO: 28 - DNA, 21 bp, synthetic construct
  misc_feature 1..21: note = 137_142_F
  source 1..21: mol_type = other DNA
cgagagacagcacgagagaaa

SEQ ID NO: 29 - DNA, 17 bp, synthetic construct
  misc_feature 1..17: note = 137_142_R
  source 1..17: mol_type = other DNA
gtgcagcagcgtgtgtc

SEQ ID NO: 30 - DNA, 27 bp, synthetic construct
  misc_feature 1..27: note = 137_142_P
  source 1..27: mol_type = other DNA
aggaggtaacggaaaaattgcaaggca

SEQ ID NO: 31 - DNA, 18 bp, synthetic construct
  misc_feature 1..18: note = IC_F
  source 1..18: mol_type = other DNA
gaatcagcccgactcgtt

SEQ ID NO: 32 - DNA, 21 bp, synthetic construct
  misc_feature 1..21: note = IC_R
  source 1..21: mol_type = other DNA
ctgagcaagtccggagataag

SEQ ID NO: 33 - DNA, 24 bp, synthetic construct
  misc_feature 1..24: note = IC_P
  source 1..24: mol_type = other DNA
ctccgactcactcctgcacgaaac

SEQ ID NO: 34 - DNA, 17 bp, synthetic construct
  source 1..17: mol_type = other DNA
acggcgattccgcagtt